(19) United States
(12) Patent Application Publication
(10) Pub. No.: US 2024/0065809 A1
(43) Pub. Date: Feb. 29, 2024

(54) BONDABLE ORTHODONTIC ASSEMBLIES AND METHODS FOR BONDING

(71) Applicant: 3M INNOVATIVE PROPERTIES COMPANY, St. Paul, MN (US)

(72) Inventors: Tsi-Zong TZOU, Afton, MN (US); Ming-Lai LAI, Afton, MN (US)

(21) Appl. No.: 18/259,636

(22) PCT Filed: Dec. 30, 2021
(86) PCT No.: PCT/IB2021/062504

§ 371 Date: Jun. 28, 2023

Related U.S. Application Data
(60) Provisional application No. 63/131,941, filed on Dec. 30, 2020.

(51) Int. Cl. A61C 7/14; A61C 7/16; A61K 6/30

(52) U.S. Cl. CPC A61C 7/14; A61C 7/16; A61K 6/30

(57) ABSTRACT

Orthodontic appliances, methods of preparing and seating said orthodontic appliances, and kits including said orthodontic appliances are described.

### BACKGROUND

Misaligned jaws and malpositioned teeth are often prevented and treated with orthodontic appliances. In many circumstances, the appliances are temporarily bonded to the tooth structure with an adhesive. When the appliance is seated against the tooth surface, excess adhesive, which is often used to fill the gap between the appliance base and tooth surface, is expressed beyond the periphery of the appliance base. The expressed adhesive, known in the art as “flash,” is manually removed by the practitioner prior to hardening; however, the procedure is time-consuming and incomplete removal can be structurally problematic. For example, accidental perturbation of the appliance during flash removal can negatively impact bond reliability. Furthermore, incomplete removal of excess adhesive is not only uncomfortable to the patient, but it also promotes bacteria accumulation which can ultimately degrade the underlying tooth structure leading to decalcification and discoloration.

Cinader previously developed a strategy for mitigating flash (see U.S. Pat. No. 10,492,890). Cinader discovered that incorporating a compressible mat at the appliance base and soaking the mat with a unfilled or lightly filled adhesive was effective to let the adhesive seep out to fill the gap between the appliance base and tooth surface and form a meniscus around the edges of the appliance upon seating the appliance, thereby eliminating the need for excess adhesive flash removal.

Despite advances, there is a continuing interest to develop appliances that bond efficiently, avoid flash removal, and mitigate manufacturing costs.

### SUMMARY

In one embodiment, an orthodontic appliance is described. The orthodontic appliance includes a base and a hardenable adhesive layer disposed on the base. The hardenable adhesive layer includes a first region and a second region, wherein the first region is at least partly surrounded by the second region, and the first region and second region are configured to contact a tooth surface. The first region includes a high-viscosity adhesive composition characterized by a viscosity of 10 Pa·s to about 1,500,000 Pa·s at a shear rate of 1 s⁻¹, and the second region includes a low-viscosity adhesive composition characterized by a viscosity of about 0.1 Pa·s to about 100 Pa·s at a shear rate of 1 s⁻¹. The high-viscosity adhesive composition has a viscosity greater than the viscosity of the low-viscosity adhesive composition.

In one embodiment, a method for seating an orthodontic appliance to a tooth surface is described. The method includes providing an orthodontic appliance described herein, contacting the orthodontic appliance to the tooth surface, applying pressure to the orthodontic appliance such that the hardenable adhesive layer compresses against the tooth surface, and hardening the hardenable adhesive layer to form a hardened adhesive.

In one embodiment, a method for preparing an orthodontic appliance described herein is described. The method includes providing a base, and forming a hardenable adhesive layer by applying a high-viscosity adhesive composition to the base at a first region, and applying a low-viscosity adhesive composition to the base at a second region. The first region is at least partly surrounded by the second region

In one embodiment, a kit is described. The kit includes an orthodontic appliance described herein and a set of instructions directing a user to perform the methods steps described herein for seating the orthodontic appliance to a tooth surface.

In one embodiment, a kit is described. The kit includes a base, a high-viscosity adhesive composition described herein, a low-viscosity adhesive composition described herein, and a set of instructions directing a user to perform the steps described herein for preparing an orthodontic appliance of the present disclosure.

### BRIEF DESCRIPTION OF DRAWINGS

FIG. 1A A side view of an orthodontic appliance of the present disclosure having a hardenable adhesive layer in an uncompressed state.

FIG. 1B A side view of an orthodontic appliance of the present disclosure having a hardenable adhesive layer in a compressed state.

FIG. 1C A bottom view of an orthodontic appliance of the present disclosure having a hardenable adhesive layer in an uncompressed state.

FIG. 1D A bottom view of an orthodontic appliance of the present disclosure having a hardenable adhesive layer in a compressed state.

FIG. 1E A top view of an orthodontic appliance of the present disclosure having a hardenable adhesive layer in an uncompressed state.

FIG. 1F A top view of an orthodontic appliance of the present disclosure having a hardenable adhesive layer in a compressed state.

FIG. 2A A cross-sectional side view of an orthodontic appliance of the present disclosure having a hardenable adhesive layer in an uncompressed state.

FIG. 2B A cross-sectional side view of an orthodontic appliance of the present disclosure having a hardenable adhesive layer in a compressed state.

FIG. 3A A cross-sectional side view of an orthodontic appliance of the present disclosure having a hardenable adhesive layer in an uncompressed state.

FIG. 3B A cross-sectional side view of an orthodontic appliance of the present disclosure having a hardenable adhesive layer in a compressed state.

FIG. 4A A cross-sectional side view of an orthodontic appliance of the present disclosure having a hardenable adhesive layer in an uncompressed state.

FIG. 4B A cross-sectional side view of an orthodontic appliance of the present disclosure having a hardenable adhesive layer in a compressed state.

FIG. 5A A side view of a comparable orthodontic appliance not of the present disclosure having a compressible mat.

FIG. 5B A side view of a comparable orthodontic appliance, not of the present disclosure, having a compressible mat.

### DETAILED DESCRIPTION

The present disclosure is directed toward an orthodontic appliance that uses an arrangement of different adhesives for providing excellent tooth adhesion while also eliminating the need for flash cleanup.

It is common practice to use adhesives to bond orthodontic appliances to teeth. The adhesive is generally coated onto the bonding surfaces of an appliance. After the appliance is mounted onto a tooth, the adhesive is hardened to produce a strong bond. A suitable orthodontic adhesive should provide a high degree of strength to maintain a consistent and secure bond between the appliances and the teeth throughout the course of orthodontic treatment, which can last for two years or more. However, the adhesive bond should not be so aggressive that the appliances become unduly difficult to remove at the end of treatment. An ideal adhesive should also have the proper degree of tack and viscosity to avoid drifting after placing the appliance on the tooth and facilitate handling by the orthodontic practitioner. Conventional orthodontic adhesives are polymerizable resins loaded with a significant amount of a hard filler, such as a quartz or silica filler. In a bonding procedure, an excess of adhesive is usually applied to the bonding surface of the appliance because the adhesive needs to serve as a gap filler (i.e., to occupy all space between the tooth surface and the appliance). In other words, when there is a mismatch between the tooth surface and the bonding surface of the appliance, the adhesive fills the space between the appliance and tooth to maintain the bond and prevent formation of voids, which can trap food and accumulate plaque. When the appliance is fully seated against the tooth, excess adhesive is expressed along the periphery of the base. This excess adhesive, known as “flash,” is manually removed by the practitioner prior to hardening the adhesive. The presence of adhesive flash can be disadvantageous. Removal of flash is time consuming, especially since accidental perturbation of the appliance can negatively impact bond reliability. Further, incomplete removal of the excess adhesive is problematic. Incomplete removal is especially common in the posterior region and behind hooked appliances, where access is limited. If not fully removed, excess adhesive provides sites for bacteria accumulation. Such bacteria can attack and degrade the underlying tooth structure, leading to decalcification and discoloration of the teeth. Further, exposed adhesive surfaces are prone to staining from food or drink. Finally, the presence of hard fillers within the adhesive composition makes removal of the adhesive, once hardened in indirect bonding cases, difficult and uncomfortable to the patient.

It is understood that adhesives having sufficient filler loading provide for better tooth adhesion, but require flash removal. Fillers increase the resistance to flow (i.e., increase viscosity), which presumably allows for more adhesive to remain in contact with the appliance-tooth junction (i.e., fill the gap between the orthodontic appliance and the tooth surface), owing at least in part to being more easily shaped into the configuration of the gap. Filling this gap is especially important for tooth structures having substantial curvatures, irregularly-shaped teeth, molars, and the like. Expressed viscous adhesive (flash), however, adopts a convex shape that must be removed, and any unremoved flash is subject to staining and creates an environment for the accumulation of caries-causing bacteria.

It is further understood that adhesives with little-to-no filler result in a softer adhesive (i.e., lower viscosity) that is expressed in a concave, meniscus-like shape (not considered “flash”) that may not need to be removed. However, low-viscosity adhesives do not adequately fill the gap between the orthodontic appliance and tooth structure, which results in poorer tooth adhesion that may lead to appliance displacement or “drift,” and may even lead to complete bond failure. Furthermore, an inadequately filled gap harbors food substances that can contribute to tooth decay. Previous solutions for adequately filling the gap between orthodontic appliances and tooth structures, while mitigating flash with low-viscosity adhesives, have been accomplished with the use of compressible mats as described in U.S. Pat. No. 10,492,890.

The inventors of the present disclosure discovered that incorporating a hardenable adhesive layer having a high-viscosity adhesive composition that is at least partly surrounded by a low-viscosity adhesive composition provides for sufficient gap fill while only allowing for the low-viscosity adhesive composition to be expressed (no flash cleanup required) upon seating the orthodontic appliance to a tooth surface. The high-viscosity adhesive composition effectively displaces the surrounding low-viscosity adhesive composition and the high-viscosity adhesive is prevented from expressing beyond the base (forming flash; requiring cleanup). Furthermore, the high-viscosity adhesive composition is distributed so as to maximize the contact area between the base and tooth structure. In addition, the adhesive layer does not flow from the base in any orientation over any period of time, which allows for full assembly and distribution. This clever construction ensures excellent bond strength without the need for flash removal, avoids use of a compressible mat and the associated manufacturing costs, and limits in-chair preparations.

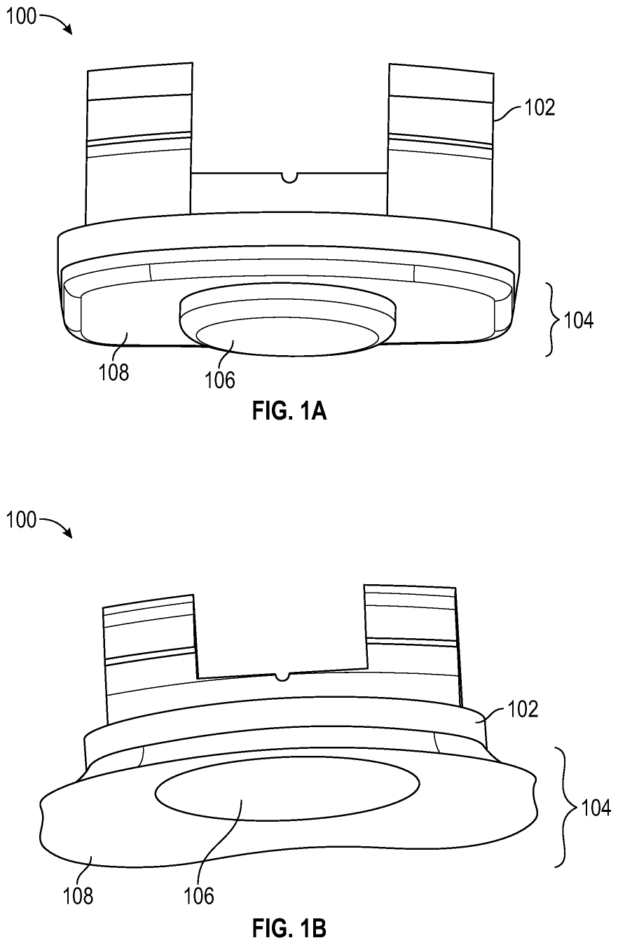

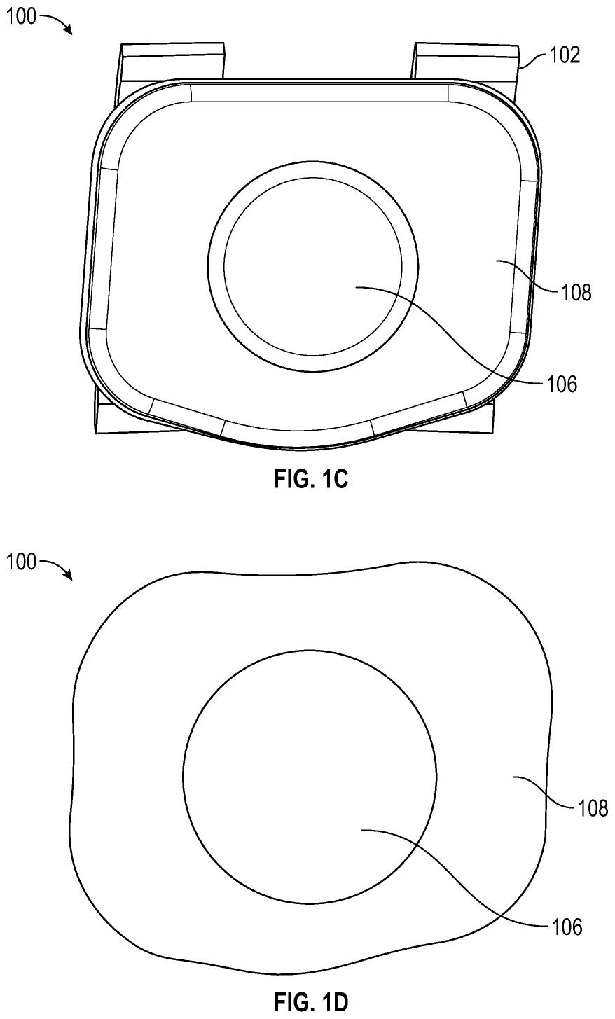

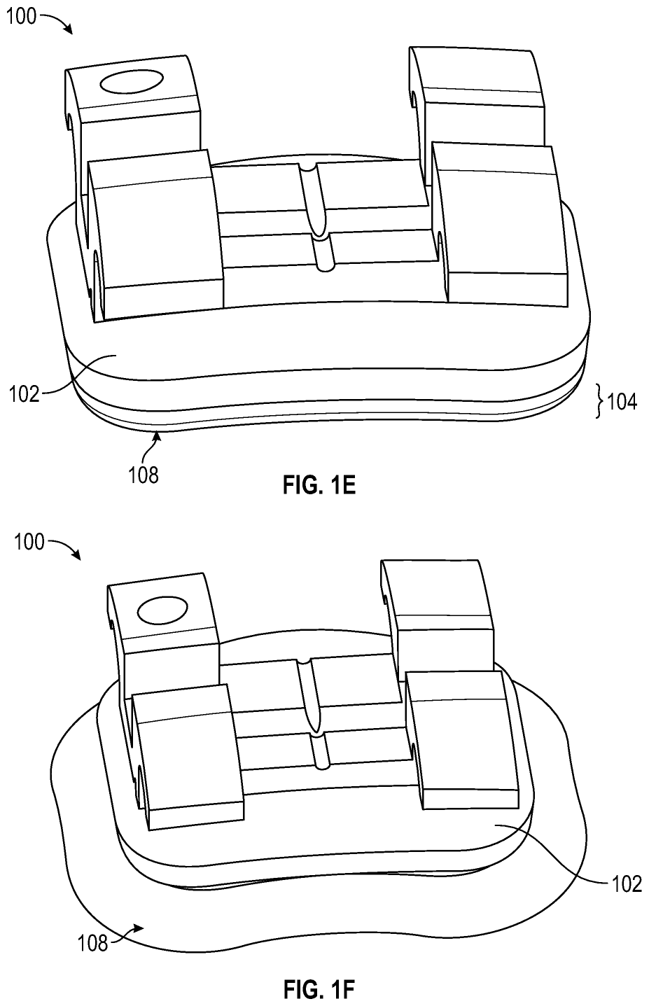

FIG. 1A illustrates a side view of orthodontic appliance 100. Orthodontic appliance 100 includes a base 102 and a hardenable adhesive layer 104 disposed thereon. Hardenable adhesive layer 104 includes a high-viscosity adhesive composition 106 surrounded by a low-viscosity adhesive composition 108. Hardenable adhesive layer 104 is depicted in an uncompressed configuration, i.e., prior to pressing orthodontic appliance 100 to a tooth surface (not shown). In one particular embodiment, orthodontic appliance 100 has high-viscosity composition 106 present in a volume of 1.24 mm³ at a maximum thickness of 0.508 mm, and covering 24% area of the base; and low-viscosity composition 108 present in a volume of 1.84 mm³ at a average thickness of 0.238 mm, and covering 76% area of the base.

FIG. 1B illustrates a side view of orthodontic appliance 100 in a compressed state, i.e., after pressing orthodontic appliance 100 to a tooth surface (not shown). As shown, only low-viscosity adhesive composition 108 extends beyond base 102. In one particular embodiment, orthodontic appliance 100, as described above in FIG. 1A, has high-viscosity composition 106 covering 47% area of the base after compression, and low-viscosity composition 108 covering 53% area of the base after compression.

FIG. 1C illustrates a bottom view of orthodontic appliance 100 in an uncompressed state.

FIG. 1D illustrates a bottom view of orthodontic appliance 100 in a compressed state.

FIG. 1E illustrates a top view of orthodontic appliance 100 in an uncompressed state.

FIG. 1F illustrates a top view of orthodontic appliance 100 in a compressed state. As shown, only low-viscosity adhesive composition 108 extends beyond base 102.

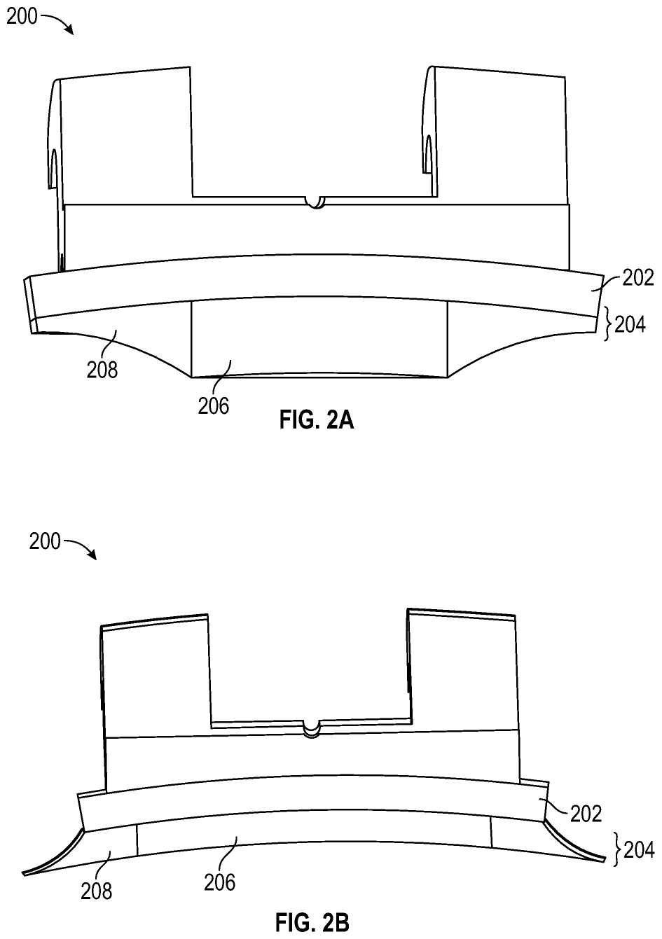

FIG. 2A illustrates a cross-sectional side view of an orthodontic appliance 200. Orthodontic appliance 200 includes a base 202 and a hardenable adhesive layer 204 disposed thereon. Hardenable adhesive layer 204 is depicted in an uncompressed configuration. As shown, the high-viscosity adhesive composition 206 has a greater volume present than low-viscosity adhesive 208. In one particular embodiment, orthodontic appliance 200 has high-viscosity composition 206 present in a volume of 1.72 mm³ at a maximum thickness of 0.508 mm, and covering 32% area of the base; and low-viscosity composition 208 present in a volume of 1.41 mm³ at a maximum thickness of 0.508 mm and an average thickness of about 0.250 mm, and covering 68% area of the base.

FIG. 2B illustrates a cross-sectional side view of orthodontic appliance 200 in a compressed state. High-viscosity adhesive composition 206 is shown to extend toward the edges of base 202 and low-viscosity adhesive composition 206 is shown to extend beyond the edges of base 202 in a concave configuration. The extent to which high-viscosity adhesive composition 204 extends toward the edges of base 202 depends at least on the volume of high-viscosity adhesive composition used. In one particular embodiment, orthodontic appliance 200, as described above in FIG. 2A, has high-viscosity composition 206 covering 66% area of the base after compression, and low-viscosity composition 208 covering 34% area of the base after compression.

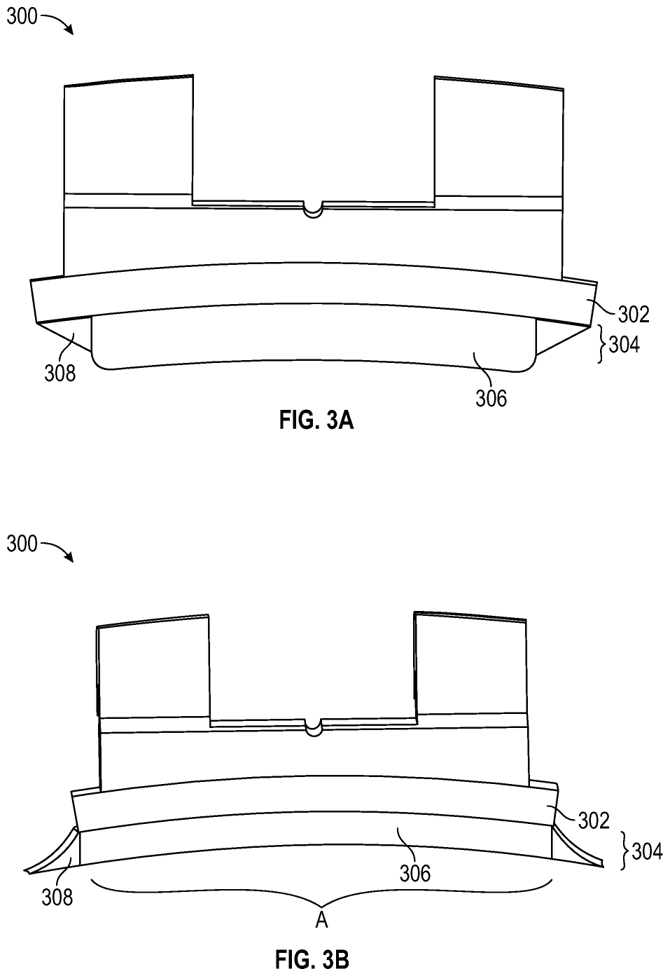

FIG. 3A illustrates a cross-sectional side view of orthodontic appliance 300, in an uncompressed state, similar to that of the orthodontic appliance 300, though differing in volume (therefore, area coverage) of high-viscosity adhesive composition 306 disposed. In one particular embodiment, orthodontic appliance 300 has high-viscosity composition 306 present in a volume of 2.67 mm³ at a maximum thickness of 0.381 mm, and covering 67% area of the base; and low-viscosity composition 308 present in a volume of 0.46 mm³ at a maximum thickness of 0.266 mm and an average thickness of 0.133 mm, and covering 33% area of the base.

FIG. 3B illustrates a cross-sectional side view of orthodontic appliance 300 in a compressed state, similar to that of the orthodontic appliance 300, though differing in the area A covered by high-viscosity adhesive composition 306. In one particular embodiment, orthodontic appliance 300, as described above in FIG. 3A, has high-viscosity composition 306 covering 100% area of the base after compression.

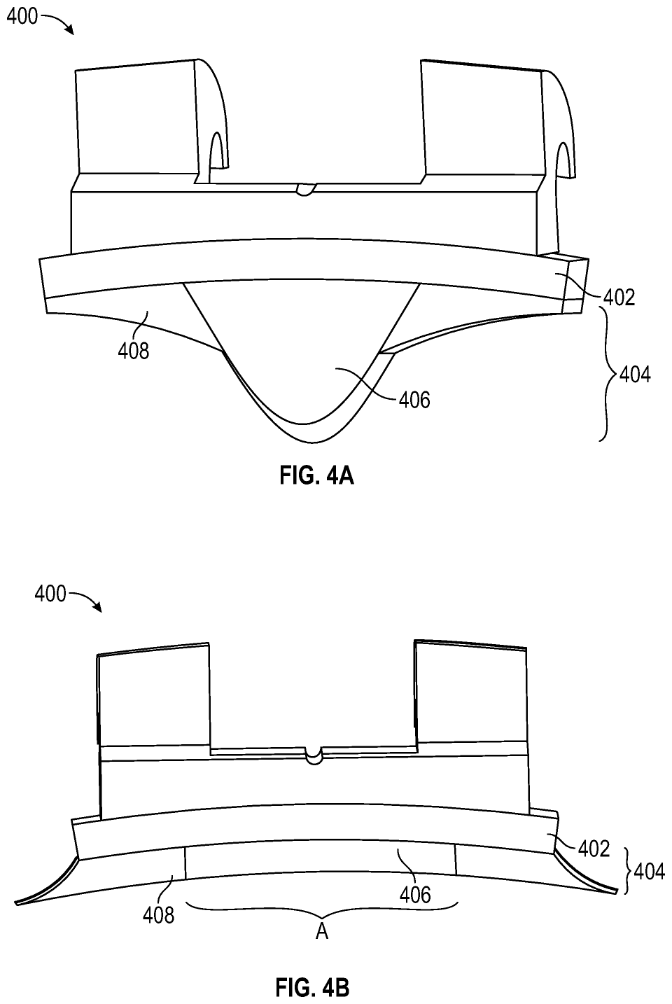

FIG. 4A illustrates a cross-sectional side view of orthodontic appliance 400, in an uncompressed state, similar to that of the orthodontic appliance 400, though differing in the height and area covered by high-viscosity adhesive composition 406. In one particular embodiment, orthodontic appliance 400 has high-viscosity composition 406 present in a volume of 1.26 mm³ at a maximum thickness of 1.15 mm, and covering 24% area of the base; and low-viscosity composition 408 present in a volume of 1.87 mm³ at a maximum thickness of 0.56 mm and an average thickness of 0.33 mm, and covering 76% area of the base.

FIG. 4B illustrates a cross-sectional side view of orthodontic appliance 400 in a compressed state, similar to that of the orthodontic appliance 200, though differing in the area A covered by high-viscosity adhesive composition 406. In one particular embodiment, orthodontic appliance 400, as described above in FIG. 4A, has high-viscosity composition 306 covering 47% area of the base after compression, and low-viscosity composition 408 covering 53% area of the base after compression.

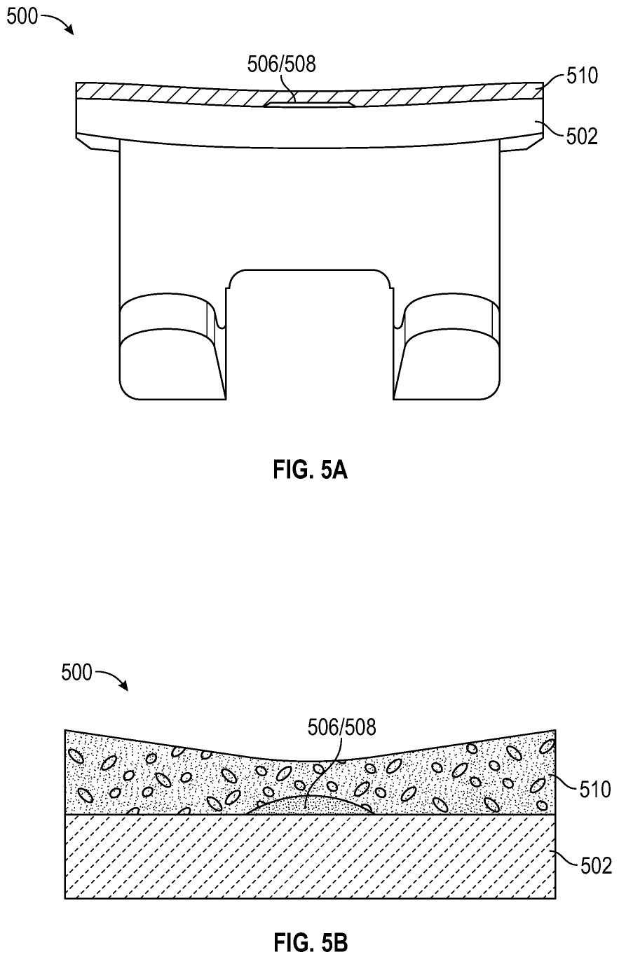

FIG. 5A illustrates an example of an orthodontic appliance 500 having a compressible material 510 and an adhesive 506/508 (high-viscosity adhesive and/or low-viscosity adhesive compositions) disposed therein and/or thereon (not part of the present disclosure; see U.S. Pat. No. 9,480,540). Orthodontic appliance 500 is shown in an uncompressed state. Compressible materials have been used to prevent adhesive compositions from extending beyond base 502 of devices. Orthodontic appliances of the present disclosure are void of such compressible materials described herein.

FIG. 5B illustrates is cross-sectional view of orthodontic appliance 500 showing compressible material 510 in an uncompressed configuration.

### Definitions

As used herein, “about” means±10 percent of a given value. For example, about 10 means 9 to 11.

As used herein, “acid functionality” refers to a functional group having an acidic hydrogen (i.e., a pKa of less than about 5). Organic acid functional groups such as —CO₂H, —P(O)(OH)₂, —S(O)₂OH, and the like, are considered “acid functionalities.”

As used herein, “alkyl” refers to a linear or branched monovalent saturated carbon chain, e.g., C₁ alkyl is methyl (—CH₃), C₂ alkyl is ethyl (—CH₂CH₃), a C₄ alkyl may be butyl (—CH₂CH₂CH₂CH₃), sec-butyl (—CH(CH₃)CH₂CH₃), iso-butyl (—CH₂CH(CH₃)₂), or tert-butyl (—C(CH₃)₃), and the like.

As used herein, “alkylene” refers to a linear or branched bivalent saturated carbon chain, e.g., C₁ alkylene is methylene (—CH₂—), C₂ alkylene in ethylene (—CH₂CH₂—), C₄ alkylene may —CH₂CH₂CH₂CH₂—, —CH(CH₃)CH₂CH₂—, —CH₂CH(CH₃)CH₂—, or —C(CH₃)₂CH₂—, and the like.

As used herein, “alkyl (alk)acrylate” refers to an alkyl acrylate ester (i.e., C(R¹)(R¹)═C(R²)C(O)O-alkyl), wherein R¹ is arbitrary (e.g., —H or C₁₋₄ alkyl) and R² is —H) or an alkyl-substituted alkyl acrylate ester, (i.e., C(R¹)(R¹)═C(R²)C(O)O-(alkyl), wherein R¹ is arbitrary (e.g., —H or C₁₋₄ alkyl) and R² is alkyl, (e.g., methyl (“meth”))). A “hydroxy-substituted (alkyl)acrylate” refers to a compound of the formula C(R¹)(R¹)═C(R²)C(O)O—R³), wherein at least one of R¹, R², or R³ is alkyl substituted with —OH. In some cases, an (alkyl)acrylate may be a dimer, e.g., C(R¹)(R¹)═C(CH₃)C(O)O—R²—OC(O)C(CH₃)═C(R¹)(R¹) wherein R² is a linking group, e.g., C₂₋₈ alkylene.

As used herein, “base” refers to the surface of an orthodontic appliance intended to contact the tooth structure. A base of an orthodontic appliance may be constructed from metal, plastic, ceramic, or a combination thereof.

As used herein, “unsaturated”, “unsaturated organic groups” refers to an olefin unit. An olefin is —C(R¹)═C(R²)—, wherein R¹ and R² is anything, e.g., each R¹ is H or C₁₋₆ alkyl; each R² is —C(O)(C₁₋₆ alkyl), —CO₂H, —O(C₁₋₆ alkyl), or the like. The term “unsaturated monomer” refers to a polymerizable compound comprising an olefin unit.

As used herein, “compressible material” or “compressible mat” refers to any non-adhesive material that reduces in volume when compressed, especially porous materials. Example compressible materials include foams (e.g., cellulose, glass, polymeric), sponges, nonwoven fabrics, glass wool, cotton fibers, and cellulose fibers. Further examples of compressible materials are disclosed in U.S. Pat. Nos. 10,492,890 and 9,480,540 and the references described therein, each of which are incorporated by reference herein in their entireties. The orthodontic appliances of the present disclosure are void of compressible materials on the base.

As used herein, the phrase “one or more of” such as used in the phrase “one or more of A and B” or “one or more of at least one A and at least one B” means a composition may include at least one A, more than one A, at least one B, more than one B, at least one A and at least one B, more than one A and more than one B. In other words, the phrase is not intended to mean the composition must have at least one of each of A and B.

As used herein, “orthodontic appliance” refers to any device intended to be bonded to a tooth structure, e.g., orthodontic brackets, buccal tubes, lingual retainers, orthodontic bands, bite openers, buttons, pre-fabricated attachments, and cleats.

As used herein, “hardenable adhesive” refers to a composition including polymerizable components (i.e., resin), with or without filler(s), that can be cured or solidified, for example, by heating to cause polymerization or chemical crosslinking.

As used herein, “hardened adhesive” refers to a composition including polymerized components, with or without filler(s), derived from a hardenable adhesive.

As used herein, “high-viscosity adhesive composition” is defined as a composition having one or more polymerizable components, wherein upon hardening, provides a polymerized adhesive. The nature of the polymerizable components (i.e., monomer(s)) alone, or in combination with additives, e.g., fillers, provides for a viscosity of about 10 Pa·s to about 1,500,000 Pa·s at a shear rate of 1 s⁻¹. A high-viscosity adhesive composition is a filled (e.g. a composite) material (e.g., a dental or orthodontic material) that are capable of being applied or adhered to a tooth surface or tooth replica surface, e.g., stone model or plaster model (i.e., for indirect bonding). High-viscosity adhesive compositions include, for example, orthodontic adhesives, cements (e.g., glass ionomer cements, resin-modified glass ionomer cements, and/or orthodontic cements), and restoratives (e.g., a restorative filling material). High-viscosity adhesive compositions may be photopolymerizable and/or redox polymerizable.

As used herein, “low-viscosity adhesive composition” is defined as a composition having one or more monomers, wherein upon hardening, provides a polymerized adhesive. The nature of the polymerizable components (i.e., monomer(s)) alone, or in combination with additives, e.g., fillers, provides for a viscosity of about 1 Pa·s to about 100 Pa·s at a shear rate of 1 s⁻¹. The low-viscosity adhesive composition is a not filled or is a lightly-filled material (e.g., a dental or orthodontic material) that is capable of being applied or adhered to a tooth surface or tooth replica surface. Low-viscosity adhesive compositions include, for example, dental adhesives, primers (e.g., orthodontic primers), liners, sealants, and coatings. Low-viscosity adhesive compositions may be photopolymerizable and/or redox polymerizable.

As used herein, a “layer” is defined as a plane parallel to the length of the base. The plane must traverse each component within the layer for the components to be considered part of the layer. The composition(s) that forms the layer may extend above and/or below said plane.

As used herein, “thickness” as defined with respect to dimensions defining an arrangement of a low-viscosity adhesive composition and a high-viscosity adhesive composition, is measured as the perpendicular distance from the base of the orthodontic appliance to the highest point of the compositions. A “maximum thickness” refers to the greatest distance measured within the region. An “average thickness” refers to the mean average of the distances measured over the entire region.

As used herein, the term “resin” refers to a polymerizable component that contains one, two, three, or more polymerizable groups. Exemplary polymerizable groups include, but are not limited to, acrylate groups, epoxy (oxirane) groups, and vinyl ether groups. A resin can often be cured by radiation induced polymerization or crosslinking, or by using a redox initiator. The terms “resin” is used synonymously with “polymerizable component(s)” and “adhesive.”

As used herein, “yield stress” is the minimum stress required to cause a material to flow.

### Orthodontic Appliances

In various embodiments, an orthodontic appliance is described. The orthodontic appliance may include a base and a hardenable adhesive layer disposed on the base. The hardenable adhesive layer may include a first region and a second region, wherein the first region is at least partly surrounded by the second region, and the first region and second region are configured to contact a tooth surface. The first region may include a high-viscosity adhesive composition characterized by a viscosity of about 10 Pa·s to about 1,500,000 Pa·s at a shear rate of 1 s⁻¹, and the second region includes a low-viscosity adhesive composition characterized by a viscosity of about 0.1 Pa·s to about 100 Pa·s at a shear rate of 1 s⁻¹. The high-viscosity adhesive composition has a viscosity greater than the viscosity of the low-viscosity adhesive composition.

In some embodiments, the orthodontic appliance consists essentially of the base and the hardenable adhesive layer.

In some embodiments, the orthodontic appliance may exclude a compressible mat. Compressible mats have been employed for filling the gap between the surface of the base and the tooth structure. Compressible materials and mats manufactured therefrom can be found, for example, in U.S. Pat. Nos. 10,492,890 and 9,480,540, each of which are incorporated herein by reference in their entireties. For example, compressible mats may include foams, sponges, nonwoven fabrics, glass wool, cotton fibers, cellulose fibers, a combination thereof, or the like. In some embodiments, the compressible materials may have a thickness of about 0.2 mm to about 1 mm, e.g., 0.5 mm. Specific materials are described, for example, in U.S. Pat. Nos. 4,605,402; 4,865,596; 5,614,570; 6,027,795; 6,645,618; Japanese Patent No. JP63170437; and Nacht et al., “The microsponge: a novel topical programmable delivery system,” in Topical Drug Delivery Formulations, D. W. Osborn and A. H. Amman (Eds.), Marel Dekker, New York, pp. 299-325 (1990), U.S. Pat. No. 5,770,636 (Wernsing et al.) and U.S. Pat. No. 5,817,704 (Shively et al.); closed-celled foams as described, for example, in Westerman et al., British Journal of Sports Medicine, 36:205-208 (2002); U.S. Pat. Nos. 6,645,618; 6,750,261, each of which are incorporated herein by reference in their entireties. For example, compressible mats may include or consist essentially of polypropylene.

### Orthodontic Appliance Base

In some embodiments, the base may have an area of about 8.0 mm² to about 20 mm².

In some embodiments, the base may be the base of an orthodontic appliance (e.g., a lingual bracket, self-ligating bracket, a Roth bracket, an MBT bracket, etc.), a buccal tube, a band, a button, a spacer maintainer, a lingual retainer, a custom base of a bracket or buccal tube, or the like.

In some embodiments, the base may include materials selected from ceramic, cobalt chromium, composite, gold, plastic, stainless steel, titanium, or a combination thereof.

### Hardenable Adhesive Layer

In some embodiments, the hardenable adhesive layer may further include fillers described herein. Fillers may be present within the high-viscosity adhesive composition, the low-viscosity adhesive composition, or a combination thereof. In many embodiments, the high-viscosity adhesive composition may include filler in an amount greater than a filler amount present in the low-viscosity adhesive composition. In some embodiments, the high-viscosity adhesive may be based on identical resin as the low-viscosity adhesive, wherein the compositions thereof differ in the amount of filler present such that the recited viscosities for the high-viscosity adhesive composition and the low-viscosity adhesive composition are achieved. In other embodiments, the high-viscosity adhesive and the low-viscosity adhesive are based on different resins, i.e., the adhesives include, or are derived from, one or more different monomers.

In some embodiments, the high-viscosity adhesive composition and the low-viscosity adhesive composition may differ in viscosity by at least 5-fold, at least 6-fold, at least 7-fold, at least 8-fold, at least 9-fold, at least 10-fold, at least 12-fold, at least 15-fold, at least 18-fold, at least 20-fold, at least 50 fold, at least 100 fold, or a value within a range between any of the preceding values, for example, between about 8 and about 12-fold, between about 10-fold and about 15-fold, or the like.

In some embodiments, the high-viscosity adhesive composition may be present on the base (in an uncompressed state) at a maximum thickness from about 0.3 mm to about 2.0 mm. For example, the high-viscosity adhesive composition may be present on the base at a maximum thickness, in mm, of about 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0, or a value within a range between any of the preceding values, e.g., between about 0.3 and about 0.5, between about 0.5 and about 1.6, or the like. In some embodiments, the high-viscosity adhesive composition may be present on the base (in an uncompressed state) at a volume from about 1.0 mm³ to about 3.0 mm³. For example, the high-viscosity adhesive composition may be present on the base at a volume, in mm³ of about 1.0, 1.2, 1.4, 1.6, 1.8, 2.0, 2.2, 2.4, 2.6, 2.8, or 3.0, or a value within a range between any of the preceding values, e.g., between about 1.2 and about 1.5, between about 1.8 and about 2.8, or the like.

In some embodiments, the low-viscosity adhesive composition may be present on the base (in an uncompressed state) at a maximum thickness from about 0.2 mm to about 1.5 mm. For example, the low-viscosity adhesive composition may be present on the base at a maximum thickness, in mm, of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0, or a value within a range between any of the preceding values, e.g., between about 0.3 and about 0.5, between about 0.5 and about 1.6, or the like. In some embodiments, the low-viscosity adhesive composition may be present on the base (in an uncompressed state) at a volume from about 0.3 mm³ to about 2.0 mm³. For example, the low-viscosity adhesive composition may be present on the base ata volume, in mm³ of about 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, or a value within a range between any of the preceding values, e.g., between about 1.4 and about 1.9, between about 0.4 and about 0.8, or the like.

In some embodiments, the ratio of maximum thickness of the high-viscosity adhesive composition (in an uncompressed state) to the average thickness of the low-viscosity adhesive composition (in an uncompressed state) may be at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, or a ratio between any of the preceding values, for example, between about 2:1 and about 4:1, between about 12:1 and about 15:1, or the like.

In some embodiments, the high-viscosity adhesive composition may cover an area of the base (in an uncompressed state) of about 20% to about 75%. For example, the high-viscosity adhesive composition may cover an area of the base (in an uncompressed state) of about 30%, 35%, 40%, 45%, 50%, 55%, 60%, 65%, 70%, 75%, or a value within a range between any of the preceding values, e.g., between about 20% and about 55%, between about 60% and about 70%, or the like.

In some embodiments, the low-viscosity adhesive composition may cover an area of the base (in an uncompressed state) of about 25% to about 80%. For example, the low-viscosity adhesive composition may cover an area of the base (in an uncompressed state) of about 25%, 30%, 35%, 40%, 45%, 50%, 55%, 60%, 65%, 70%, 75%, or 80%, or a value within a range between any of the preceding values, e.g., between about 30% and about 40%, between about 45% and about 80%, or the like. The low-viscosity adhesive composition may cover the difference of the area of the base not occupied by the high-viscosity adhesive composition.

In some embodiments, the total area of the base may be covered by the first region (including the high-viscosity adhesive composition in an uncompressed state) and the second region (including the low-viscosity adhesive composition in an uncompressed state) at a ratio of about 1:1, 1:1.2, 1:1.4, 1:1.6, 1:1.8, 1:2, 1:2.2, 1:2.4, 1:2.6, 1:2.8, 1:3, 1:3.2, 1:3.4, 1:3.6; 1:3.8, 1:4, 1:4.2, 1:4.4, 1:4.6, 1:4.8, 1:5, or a ratio between any of the preceding values, for example, between about 1:1.6 and about 1:2.5, between about 1:3 and about 1:4, or the like.

In some embodiments, the high-viscosity adhesive composition may cover an area of the base (after compression) of about 40% to about 100%. For example, the high-viscosity adhesive composition may cover an area of the based (after compression) of about 40%, 45%, 50%, 55%, 60%, 65%, 70%, 75%, 80%, 85%, 90%, 95%, or 100%, or a value within a range between any of the preceding values, e.g., between about 45% and about 70%, between about 50% and about 95%, or the like.

In some embodiments, the low-viscosity adhesive composition may cover an area of the base (after compression) of about of about 0% to about 60%. For example, the low-viscosity adhesive composition may cover an area of the base (after compression) of about 0%, 5%, 10%, 15%, 20%, 25%, 30%, 35%, 40%, 45%, 50%, 55%, or 60%, or a value within a range between any of the preceding values, e.g., between about 5% and about 50%, between about 30% and about 55%, or the like. The low-viscosity adhesive composition may cover the difference of the area of the base not occupied by the high-viscosity adhesive composition.

In some embodiments, the high-viscosity adhesive composition and the low-viscosity adhesive composition may be selected from any compositions described herein, including any filler loadings, and any combination of compositions thereof. For example, the high-viscosity adhesive may be 3M Transbond™ XT Adhesive or 3M Transbond™ PLUS Color Change Adhesive and the low-viscosity adhesive may be 3M Transbond™ XT Primer, 3M Clinpro Sealant or 3M Scotchbond Universal Adhesive.

In some embodiments, at least one of the high-viscosity and low-viscosity adhesives within the hardenable adhesive layer may further include an initiator system. The initiator system may be present within the low-viscosity adhesive composition, the high-viscosity adhesive composition, or a combination thereof. In some embodiments, the initiator system may include one or more photoinitiator. A photoinitiator initiates polymerization (hardening) of the adhesive composition(s). Examples of photoinitiators are described in U.S. Pat. Nos. 5,545,676 and 7,674,850, 7,816,423, e.g., diaryliodonium salt, metal complex salt, or the like. Other example polymerization initiators include ketones, e.g., benzyl, benzoin, acyloins, acyloin ethers, or the like. Specific examples of polymerization intiators include 2,2-dimethoxy-2-phenylacetophenone ((i.e., IRGACURE 651) and 2-methoxy-2-phenylacetophenone (Ciba Specialty Chemicals Corp., Tarrytown, NY). In some embodiments, the photoinitiator may be present in the hardenable adhesive layer in an amount of about 0.01 wt % to about 10 wt % with respect to the weight of the hardenable adhesive layer. For example, the photoinitiator may be present in an amount in wt % with respect to the weight of the hardenable adhesive layer of about 0.01, 0.05, 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0, or a value between any of the preceding values, e.g., between about 5.0 and about 8.0, between about 0.1 and about 3.5, or the like.

In other embodiments, the initiator system may include a redox initiator. The redox initiator may include one or more redox agents, i.e., reducing agent and oxidizing agent. Reducing agents and oxidizing agents may react to produce free-radical species capable of initiating polymerization. Suitable redox agents can be found, for example, in U.S. Pat. Nos. 7,173,074 and 6,982,288. For example, ascorbic acid and salts, derivatives, or metal complexes thereof are suitable reducing agents, as are amines (e.g., 4-tert-butyl dimethylaniline; p-toluenesulfinic salts and benzenesulfinic salts), thioureas (e.g., 1-ethyl-2-thiourea, tetraethyl thiourea, tetramethyl thiourea, 1,1-dibutyl thiourea, and 1,3-dibutyl thiourea), cobalt (II) chloride, ferrous chloride, ferrous sulfate, hydrazine, hydroxylamine, sulfite and dithionite salts, or the like. Suitable oxidizing agents include, for example, persulfuric acid and salts thereof, akyl ammonium salts, peroxide (e.g., benzoyl peroxides, hydroperoxides such as cumyl hydroperoxide, t-butyl hydroperoxide, amyl hydroperoxide), transition metal salts such as cobalt (III) chloride, ferric chloride, cerium (IV) sulfate, perboric acid and salts thereof, permanganic acid and salts thereof, perphosphoric acid and salts thereof, or the like. Enzymes, such as those disclosed in U.S. Pat. Pub. No. 2004/0122126, may also serve as redox initiators.

In some embodiments, the initiator system may further include one or more sensitizer (e.g., ketones, coumarin dyes, xanthene dyes, acridine dyes, thiazole dyes, thiazine dyes, oxazine dyes, azine dyes, aminoketone dyes, porphyrins, aromatic polycyclic hydrocarbons, p-substituted aminostyryl ketone compounds, aminotriaryl methanes, merocyanines, squarylium dyes, pyridinium dyes, or the like). Specific examples of sensitizers include camphorquinone, glyoxal, biacetyl, 3,3,6,6-tetramethylcyclohexanedione, 3,3,7,7-tetramethyl-1,2-cycloheptanedione, 3,3,8,8-tetramethyl-1,2-cyclooctanedione, 3,3,18,18-tetramethyl-1,2-cyclooctadecanedione, dipivaloyl, benzil, furil, hydroxybenzil, 2,3-butanedione, 2,3-pentanedione, 2,3-hexanedione, 3,4-hexanedione, 2,3-heptanedione, 3,4-heptanedione, 2,3-octanedione, 4,5-octanedione, 1,2-cyclohexanedione, or the like. In some embodiments, the initiator system may further include one or more electron donor, e.g., amines, amides, ethers, thioethers, ureas, thioureas, ferrocene, sulflnic acids or salts thereof, ferrocyanide salts, ascorbic acid or its salts, dithiocarbamic acid or its salts, xanthate salts, ethylene diamine tetracetic acid salts, tetraphenylboronic acid salts, or the like. In some embodiments, the initiator system may further include one or more hydrogen donor, e.g., amines.

In some embodiments, the hardenable adhesive layer may further include glass ionomer cements, resin-modified glass ionomer cements, or a combination thereof.

In some embodiments, at least one of the high-viscosity and low-viscosity adhesives within the hardenable adhesive layer may further include one or more fluoride-releasing agent. Incorporating one or more fluoride-releasing agent within the high-viscosity adhesive composition and/or low-viscosity adhesive composition may allow for fluoride deliver to the tooth surface beneath and immediately around the orthodontic appliance, thereby protecting the tooth surface from decay. Examples of fluoride-releasing agents include fluoroaluminosilicate glass, inorganic fluoride salts, organic fluoride salts, fluoride-containing metal complexes, or the like. Specific fluoride-releasing agents may be found, for example, in the following disclosures: U.S. Pat. Nos. 3,814,717, 5,332,429, 6,126,922, and Int. Pat. Pub. No. WO 2000/69393. In some embodiments, the hardenable adhesive layer may include a fluoride-releasing agent present in an amount of about 0.1 wt % to about 85 wt % with respect to the weight of the hardenable adhesive layer. For example, the fluoride-releasing agent may be present in an amount in wt % with respect to the weight of the hardenable adhesive layer of about 0.1, 0.5, 1, 5, 10, 15, 20, 25, 30, 35, 40, 45, 50, 55, 60, 65, 70, 75, 80, or 85, or a value within a range between any of the preceding values, e.g., between about 20 and about 30, between about 45 and about 60, or the like.

### Adhesive Compositions

In many embodiments, the high-viscosity adhesive composition includes a resin and a filler(s) described herein. In many embodiments, the low-viscosity adhesive composition includes a resin and optionally a filler(s) described herein. The high-viscosity adhesive resin and the low-viscosity adhesive resin may include the same or different polymerizable components, wherein the compositions having the same resin may differ based on the type and/or amount of filler.

In some embodiments, the high-viscosity adhesive resin and low-viscosity adhesive resin may independently include one or more polymerizable components with or without acid functionality, or a combination thereof.

Polymerizable components without acid functionality may include, for example, PEGDMA (polyethyleneglycol dimethacrylate having a molecular weight of approximately 400), bisGMA, UDMA (urethane dimethacrylate), ODMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Other examples include epoxy resins, such as those listed in U.S. Pat. No. 6,187,836 (Oxman et al.) and U.S. Pat. No. 6,084,004 (Weinmann et al.), U.S. Pat. No. 6,245,828 (Weinmann et al.) U.S. Pat. No. 5,037,861 (Crivello et al), and U.S. Pat. No. 6,779,656 (Klettke et al.), U.S. Pat. No. 3,018,262 (Schroeder), WO 01/51540 (Klettke et al.). U.S. Pat. No. 7,262,228 (Oxman et al.) and in “Handbook of Epoxy Resins” by Lee and Neville, McGraw-Hill Book Co., New York (1967). 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexyl-methyl) adipate, octadecylene oxide, epichlorohydrin, styrene oxide, vinyl cyclohexene oxide, glycidol, glycidylmethacrylate, diglycidyl ether of Bisphenol A. The references are incorporated herein in their entireties.

Polymerizable components with acid functionality may include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl) phosphate, ((meth)acryloxypropyl) phosphate, bis((meth)acryloxypropyl) phosphate, bis((meth)acryl oxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof. Certain of these compounds are obtained, for example, as reaction products between isocyanatoalkyl (meth)acrylates and carboxylic acids. Additional compounds of this type having both acid-functional and ethylenically unsaturated components are described in U.S. Pat. No. 4,872,936 (Engelbrecht) and U.S. Pat. No. 5,130,347 (Mitra). polymerizable bisphosphonic acids as disclosed for example, in U.S. Patent Publication No. 2004/0206932 (Abuelyaman et al.); AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mita)); and those recited in U.S. Pat. No. 4,259,075 (Yamauchi et al.), U.S. Pat. No. 4,499,251 (Omura et al.). U.S. Pat. No. 4,537,940 (Omura et al.), U.S. Pat. No. 4,539,382 (Omura et al.), U.S. Pat. No. 5,530,038 (Yamamoto et al.). U.S. Pat. No. 6,458,868 (Okada et al.), and European Patent Application Publication Nos. EP 712,622 (Tokuyama Corp.) and EP 1,051,961 (Kuraray Co., Ltd.). Further examples of polymerizable components with acid functionality include (meth)acryloxy group and at least one —O—P(O)(OH)ₓ group, wherein x=1 or 2, and wherein the at least one —O—P(O)(OH)ₓ group and the at least one (meth)acryloxy group are linked together by a C1-C4 hydrocarbon group; a second compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C5-C12 hydrocarbon group: an ethylenically unsaturated compound without acid functionality: an initiator system; and a filler. Such compositions are described, for example, in Published U.S. Application No. 2007/0248927 (Luchterhandt et al.). See, also, U.S. Pat. No. 7,449,499 (Bradley et al.) and U.S. Pat. No. 7,452,924 (Aasen et al.); and Published U.S. Application Nos. 2005/0175966 (Falsafi et al.), 2009/0011388 (Bradley et al.), and 2009/0035728 (Aasen et al.) The references are incorporated herein in their entireties.

In some embodiments, the high-viscosity adhesive resin and low-viscosity adhesive resin may independently include polymerizable components selected from an unsaturated monomer (e.g., a (meth)acrylate, an epoxy (meth)acrylate, a hydroxy-substituted (meth)acrylate, a (meth)acrylic acid, a hydroxy-substituted (meth)acrylic acid, a vinyl ether), an epoxy resin, or the like. For example, the one or more unsaturated monomer may be selected from methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenol A di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), bisphenol A bis(2-hydroxyethyl ether) dimethacrylate, 2-hydroxy-1,2,3-propanetricarboxylic acid, CDMA (reaction product of 2-hydroxy-1,2,3-propanetricarboxylic acid and 2-isocyanatoethyl methacrylate), decamethylene dimethacrylate, methacryloxydecyl phosphate (MDP) and other acryl phosphates, acrylic acid and other acryl acids, copolymers of acrylic acid and itaconic acid, copolymerizable mixtures of acrylated monomers such as those in U.S. Pat. No. 4,652,274 (Boettcher et al.), acrylated oligomers such as those of U.S. Pat. No. 4,642,126 (Zador et al.), and polyunsaturated carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.), hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-ethacryloxypropoxy)phenyl]propane (bisGMA), PEGDMA (polyethyleneglycol dimethacrylate having a molecular weight of approximately 400), UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). The references are incorporated herein in their entireties.

In some embodiments, the high-viscosity adhesive resin and low-viscosity adhesive resin may independently include one or more polymerizable components independently selected from bisphenol A bis(2-hydroxyethyl ether) dimethacrylate, bisphenol A diglycidyl ether dimethacrylate, CDMA (citric acid dimethacrylate, reaction product of 2-hydroxy-1,2,3-propanetricarboxylic acid and 2-isocyanatoethyl methacrylate), polyethylene glycol dimethacrylate, triethylene glycol dimethacrylate, 2-hydroxyethyl methacrylate, decamethylene dimethacrylate, methacryloxydecyl phosphate, dimethylaminoethyl methacrylate, and copolymer of acrylic acid and itaconic acid.

In some embodiments, the high-viscosity adhesive resin may include one or more polymerizable components selected from bisphenol A bis(2-hydroxyethyl ether) dimethacrylate, bisphenol A diglycidyl ether dimethacrylate, CDMA (citric acid dimethacrylate, reaction product of 2-hydroxy-1,2,3-propanetricarboxylic acid and 2-isocyanatoethyl methacrylate), and polyethylene glycol dimethacrylate.

In some embodiments, the high-viscosity adhesive resin may include one or more polymerizable components selected from bisphenol A bis(2-hydroxyethyl ether) dimethacrylate and bisphenol A diglycidyl ether dimethacrylate.

In some embodiments, the low-viscosity adhesive resin may include one or more polymerizable components selected from bisphenol A bis(2-hydroxyethyl ether) dimethacrylate, bisphenol A diglycidyl ether dimethacrylate, triethylene glycol dimethacrylate, 2-hydroxyethyl methacrylate, decamethylene dimethacrylate, methacryloxydecyl phosphate, dimethylaminoethyl methacrylate, and copolymer of acrylic acid and itaconic acid.

In some embodiments, the low-viscosity adhesive resin may include one or more polymerizable components selected from bisphenol A bis(2-hydroxyethyl ether) dimethacrylate, and bisphenol A diglycidyl ether dimethacrylate.

In some embodiments, the low-viscosity adhesive resin may include one or more polymerizable components selected from triethylene glycol dimethacrylate and bisphenol A diglycidyl ether dimethacrylate.

In some embodiments, the low-viscosity adhesive resin may include one or more polymerizable components selected from 2-hydroxyethyl methacrylate, bisphenol A diglycidyl ether dimethacrylate, decamethylene dimethacrylate, methacryloxydecyl phosphate, dimethylaminoethyl methacrylate, and copolymer of acrylic acid and itaconic acid.

In some embodiments, the low-viscosity adhesive resin may include water, alcoholic solvents, e.g., ethanol, or a combination thereof.

In some embodiments, the high-viscosity adhesive composition may include one or more adhesive compositions sold under the names, Transbond XT Adhesive (3M Unitek, Monrovia, CA), Transbond PLUS Color Change Adhesive (3M Unitek, Monrovia, CA), and Transbond Supreme LV Adhesive (3M Unitek, Monrovia, CA), including adhesives within devices thereof. In other embodiments, the low-viscosity adhesive composition may include one or more adhesive compositions sold under the names Transbond XT Primer (3M Unitek, Monrovia CA), Clinpro Sealant (3M ESPE, St. Paul, MN), and Scotchbond Universal Adhesive (3M ESPE, St. Paul, MN), including adhesives within devices thereof. Alternatively, the high-viscosity adhesive composition and the low-viscosity adhesive composition may independently include one or more resins within the aforementioned adhesive compositions, albeit differing in filler type and/or filler amount.

In some embodiments, the high-viscosity adhesive composition may include one or more fillers described herein present in an amount of about 50 wt % to about 90 wt % with respect to the weight of the high-viscosity adhesive composition. For example, the high-viscosity adhesive composition may include filler in an amount in wt % with respect to the weight of the high-viscosity adhesive composition of about 50, 52, 54, 56, 58, 60, 62, 64, 66, 68, 70, 72, 74, 76, 78, 80, 82, 84, 86, 88, 90, or a value within a range of any of the preceding values, e.g., between about 50 and about 86, between about 70 and about 80, or the like.

In some embodiments, the low-viscosity adhesive composition may include one or more fillers described herein present in an amount of about 0 wt % to about 65 wt % with respect to the weight of the low-viscosity adhesive composition. For example, the low-viscosity adhesive composition may include filler in an amount in wt % with respect to the weight of the low-viscosity adhesive composition of about 0, 5, 10, 15, 20, 25, 30, 35, 40, 45, 50, 55, 60, or 65, or a value within a range between any of the preceding values, e.g., between about 25 and about 35, between about 10 and about 20, or the like.

In some embodiments, the high-viscosity adhesive composition may be characterized by a viscosity of about 10 Pa·s to about 1,500,000 Pa·s at a shear rate of 1 s⁻¹. For example, the high-viscosity adhesive composition may be characterized by a viscosity in Pa·s at a shear rate of 1 s⁻¹ of about 10, 50, 100, 200, 300, 400, 500, 600, 700, 800, 900, 1000, 1500, 2000, 2500, 3000, 4000, 5000, 6000, 7000, 8000, 9000, 10000, 12000, 13000, 14000, 15000, 20000, 40000, 60000, 80000, 100000, 200000, 300000, 400000, 500000, 600000, 700000, 800000, 900000, 1000000, 1500000 or a value within a range between any of the preceding values, for example between about 500 and about 1000, between about 10000 and about 100000, or the like. In some embodiments, the high-viscosity adhesive composition may be characterized by a viscosity of about 1500 Pa·s to about 5000 Pa·s at a shear rate of 1 s⁻¹. For example, the high-viscosity adhesive composition may be characterized by a viscosity in Pa·s at a shear rate of 1 s⁻¹ of about 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000, or a value within a range between any of the preceding values, e.g., between about 1800 and about 1900, between about 4000 and about 4100, or the like. In some embodiments, the high-viscosity adhesive composition may be characterized by a viscosity of about 4000 Pa·s to about 8000 Pa·s at a shear rate of 0.1 s⁻¹. For example, the high-viscosity adhesive composition may be characterized by a viscosity in Pa·s at a shear rate of 0.1 s⁻¹ of about 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, or 8000, or a value within a range between any of the preceding values, e.g., between about 5000 and about 7000, or the like. In some embodiments, the high-viscosity adhesive composition may be characterized by a viscosity of about 300 Pa·s to about 2000 Pa·s at a shear rate of 10 s⁻¹. For example, the high-viscosity adhesive composition may be characterized by a viscosity in Pa·s at a shear rate of 10 s⁻¹ of about 300, 400, 500, 600, 700, 800, 900, 1000, 1100, 1200, 1300, 1400, 1500, 1600, 1700, 1800, 1900, or 2000, or a value within a range between any of the preceding values, e.g., between about 400 and about 600, between about 1500 and about 1700, or the like.

In some embodiments, the low-viscosity adhesive composition may be characterized by a viscosity of about 0.1 Pa·s to about 100 Pa·s at a shear rate of 1 s⁻¹. For example, the low-viscosity adhesive composition may be characterized by a viscosity in Pa·s at a shear rate of 1 s⁻¹ of about 0.1, 0.5, 1.0, 5.0, 10, 15, 20, 25, 30, 35, 40, 45, 50, 55, 60, 65, 70, 75, 80, 85, 90, 95, 100, or a value within a range between any of the preceding values, e.g., between about 40 and about 100, between about 1 and about 5, between about 0.1 and about 1, or the like. In some embodiments, the low-viscosity adhesive composition may be characterized by a viscosity of about 0.5 Pa·s to about 50 Pa·s at a shear rate of 1 s⁻¹. For example, the low-viscosity adhesive composition may be characterized by a viscosity in Pa·s at a shear rate of 1 s⁻¹ of about 0.5, 0.6, 0.7, 0.8, 0.9, 1, 2, 4, 6, 8, 10, 12, 14, 16, 18, 20, 22, 24, 26, 28, 30, 32, 34, 36, 38, 40, 42, 44, 46, 68, or 50, or a value within a range between any of the preceding values, e.g., between about 0.7 and about 1, between about 30 and about 40, or the like. In some embodiments, the low-viscosity adhesive composition may be characterized by a viscosity of about 0.5 Pa·s to about 50 Pa·s at a shear rate of 0.1 s⁻¹. For example, the low-viscosity adhesive composition may be characterized by a viscosity in Pa·s at a shear rate of 0.1 s⁻¹ of about 0.5, 0.6, 0.7, 0.8, 0.9, 1, 2, 4, 6, 8, 10, 12, 14, 16, 18, 20, 22, 24, 26, 28, 30, 32, 34, 36, 38, 40, 42, 44, 46, 68, or 50, or a value within a range between any of the preceding values, e.g., between about 0.7 and about 1, between about 30 and about 40, or the like In some embodiments, the low-viscosity adhesive composition may be characterized by a viscosity of about 0.5 Pa·s to about 50 Pa·s at a shear rate of 10 s⁻¹. For example, the low-viscosity adhesive composition may be characterized by a viscosity in Pa·s at a shear rate of 10 s⁻¹ of about 0.5, 0.6, 0.7, 0.8, 0.9, 1, 2, 4, 6, 8, 10, 12, 14, 16, 18, 20, 22, 24, 26, 28, 30, 32, 34, 36, 38, 40, 42, 44, 46, 68, or 50, or a value within a range between any of the preceding values, e.g., between about 0.7 and about 1, between about 20 and about 30, or the like.

In some embodiments, one or more of the high-viscosity adhesive composition and low-viscosity adhesive composition may further include a filler(s) described herein. A skilled artisan would understand how to adjust the viscosity of a composition by including viscosity-modifying fillers.

In some embodiments, the high-viscosity adhesive composition may be a paste. A paste is defined herein as a viscous mass of solids dispersed in a liquid that can be shaped before hardening. In some embodiments, the high-viscosity adhesive composition may be characterized by a yield stress of at least 1000 dynes/cm² as determined by the method described in Rheology Principles, Measurements, and Applications, C.I. W. See CW Macosko, VCH Publishers, Inc., New York, 1994, p. 92. Measurement of the yield stress and viscosity of adhesive test samples may be performed with appropriate equipment such as Rheometrics ARES controlled strain rheometer (Advanced Rheometric Expansion System, Rheometric Scientific, Inc., Piscataway, N.J.). In some embodiments, the high-viscosity adhesive composition may be characterized by a yield stress of about 1000 dynes/cm² to about 10000 dynes/cm². For example, the high-viscosity adhesive composition may be characterized by a static yield stress at 28° C. in dynes/cm² of about 1000, 2000, 3000, 4000, 5000, 6000, 7000, 7200, 7400, 7600, 7800, 8000, 8200, 8400, 8600, 8800, 9000, 9200, 9400, 9600, 9800, 10000, or a value within a range between any of the preceding values, for example, between about 7500 and about 8000, between about 7400 and about 9200, or the like.

In some embodiments, the low-viscosity adhesive composition is a flowable solution or flowable suspension. It is able to flow with moderate force and cannot be shaped prior to hardening. While the low-viscosity adhesive compositions described herein are flowable, said low-viscosity adhesives do not flow significantly from a vertically-held base as determined by the Vertical Flow Test (see Example 4) where the difference of the thicknesses of the adhesive composition at the top and at the bottom is less than about 15% after holding the base vertically for six hours at room temperature (23° C.) and five minutes at 40° C., due to surface tension within the hardenable adhesive layer configuration in contact with the base.

### First Region

In many embodiments, the first region may include any high-viscosity adhesive composition described herein.

In some embodiments, the first region may include high-viscosity adhesive composition having a maximum thickness of about 0.3 mm to about 1.5 mm. For example, the high-viscosity adhesive composition may have a maximum thickness in mm of about 0.3, 0.32, 0.34, 0.36, 0.38, 0.40, 0.42, 0.44, 0.46, 0.48, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, or a value within a range of any of the preceding values, for example, between about 0.38 and about 1.2, between about 0.60 and about 0.90, or the like.

In some embodiments, the first region may include the high-viscosity adhesive composition having a volume that is about 40 percent to about 85 percent of the total volume of the hardenable adhesive layer. For example, the volume of the high-viscosity adhesive composition may be in percent relative to the total volume of the hardenable adhesive layer of about 40, 45, 50, 55, 60, 65, 70, 75, 80, 85, or a value within a range between any of the preceding values, e.g., between about 45 and about 55, between about 60 and about 75, or the like.

In some embodiments, the first region may extend over about 20 percent to about 50 percent of the total area of the base. For example, the first region, including the high-viscosity adhesive composition, may extend over a percentage of the total area of the base of about 20, 22, 24, 26, 28, 30, 32, 34, 36, 38, 40, 42, 44, 46, 48, 50, or a value between any of the preceding values, for example, between about 30 and about 40, between about 26 and about 48, or the like.

In some embodiments, the first region may include the high-viscosity adhesive composition in a shape representing a gaussian curve, a triangle, or a trapezoid when viewed from a cross-sectional side view of the base.

In some embodiments, the first region may include the high-viscosity adhesive composition in a shape representing a circle, a polygon, or a quadrilateral when viewed from a direction perpendicular to the base.

### Second Region

In many embodiments, the second region may include any low-viscosity adhesive composition described herein.

In some embodiments, the second region may include the low-viscosity adhesive composition having an average thickness of about 0.05 mm to about 0.20 mm. For example, the low-viscosity adhesive composition may have an average thickness in mm of about 0.05, 0.06, 0.07, 0.08, 0.09, 0.10, 0.12, 0.14, 0.16, 0.18, 0.20, or a value within a range between any of the preceding values, for example between about 0.06 and about 0.16, between about 0.10 and about 0.14, or the like.

In some embodiment, the second region may include the low-viscosity adhesive composition extending over about 50 percent to about 80 percent of the total area of the base. For example, the second region, including the low-viscosity adhesive composition, may extend over a percentage of the total area of the base of about 50, 55, 60, 65, 70, 75, 80, or a value within a range between any of the preceding values, e.g., between about 60 and about 70, between about 55 and about 75, or the like.

In some embodiments, the second region may include the low-viscosity adhesive composition having a shape representing an annulus or rectangular annulus when viewed from a direction perpendicular to a plane defined by the base.

### Fillers

In some embodiments, the filler may be an inorganic material selected from non-acid reactive (e.g., quartz, submicron silica, zirconia, submicron zirconia, non-vitreous microparticles), acid reactive (e.g., metal oxides (e.g., barium oxide, calcium oxide, magnesium oxide, zinc oxide), glasses (e.g., borate glasses, phosphate glasses, fluoroaluminosilicate glasses), metal salts), and a combination thereof.

In some embodiments, the filler may be silane-treated glass, silane-treated quartz, silane-treated fumed silica, silane treated silica, silane-treated zirconia, silane-treated ceramic, or a combination thereof.

In some embodiments, the surface of the filler may be treated with a coupling agent in order to enhance the bond between the filler and the adhesive polymer (resin). Suitable coupling agents include, for example, gamma-methacryloxypropyltrimethoxysilane, gamma-mercaptopropyltriethoxysilane, gamma-aminopropyltrimethoxysilane, or the like.

In some embodiments, the filler may have a unimodal or polymodal particle size distribution. The maximum particle size may be less than 30 μm. For example, the maximum particle size in μm may be less than 30, less than 25, less than 20, less than 15, less than 10, less than 5, less than 1, less than 0.5, less than 0.1, less than 0.075, less than 0.05, less than 0.025 or a value within a range between any of the preceding values, for example, between about 0.025 and about 0.5, between about 5 and about 1, between about 30 and about 10, or the like.

In some embodiments, the filler may be selected from quartz, silica, silica nitride, feldspar, borosilicate glass, kaolin, talc, zirconia, titania, glasses derived from Zr, Sr, Ce, Sb, Sn, Ba, Zn, Al, feldspar, submicron silica particles such as those available under the trade designations AEROSIL (e.g., OX 50), silicas from Degussa Corp (Akron, OH), silicas from Cabot Corp (Tuscola, IL), pulverized polycarbonate, methacrylates of polycaprolactone, polyepoxides, metal oxides (e.g., barium oxide, calcium oxide, magnesium oxide, zinc oxide), glasses (borate glasses, phosphate glasses, fluoroaluminosilicate glasses, metal salts, non-vitreous microparticles such as those described in U.S. Pat. No. 4,503,169 (incorporated herein by reference in its entirety), low Mohs fillers such as those described in U.S. Pat. No. 5,695,251 (incorporated herein by reference in its entirety), or a combination thereof. Other suitable fillers are described in U.S. Pat. Nos. 6,387,981; 6,572,693; International Pub. Nos. WO 01/30305; WO 01/30306; WO 01/30307; and WO 03/063804 (each of which are incorporated herein by reference in their entireties). Suitable nanofiller are described in U.S. Pat. Nos. 7,090,721; 7,090,722; 7,156,911; and U.S. Pat. Pub. No. 2005/0256223 (each of which are incorporated herein by reference in their entireties).

In some embodiments, the high-viscosity adhesive composition may include one or more filler present in a total amount of about 1 wt % to about 85 wt % based on the weight of the high-viscosity adhesive composition. For example, the high-viscosity adhesive composition may include one or more filler present in a total amount, in wt %, of about 1, 5, 10, 15, 20, 25, 30, 35, 40, 45, 50, 55, 60, 65, 70, 75, 80, or 85, or a value within a range between any of the preceding values, e.g., between about 50 and about 75, between about 25 and about 60, or the like. In some embodiments, the high-viscosity adhesive composition may include silane-treated glass, silane-treated quartz, silane-treated silica, or a combination thereof.

In some embodiment, the low-viscosity adhesive composition may include one or more filler present in a total amount of about 0 wt % to about 50 wt %. For example, the low-viscosity adhesive may include one or more filler present in a total amount, in wt %, of about 0, 0.2, 0.5, 1, 2, 3, 4, 5, 6, 7, 8, 9, 10, 11, 12, 13, 14, 15, 16, 17, 18, 19, 20, 25, 30, 40, 50, ora value within a range between any of the preceding values, e.g., between about 1 and about 5, between about 2 and about 3, or the like. In some embodiments, the low-viscosity adhesive composition may include silane-treated ceramic, silane-treated silica, titanium dioxide, or a combination thereof.

### Methods for Seating Orthodontic Appliances

In various embodiments, a method for seating an orthodontic appliance to a tooth surface is described. The method may include providing an orthodontic appliance described herein, contacting the orthodontic appliance to the tooth surface, applying pressure to the orthodontic appliance such that the hardenable adhesive layer compresses against the tooth surface, and hardening the hardenable adhesive layer to form a hardened adhesive.

In some embodiments, the method may further include applying one or more orthodontic appliance to a prefabricated tray prior to contacting the orthodontic appliance(s) to the tooth surface(s). The prefabricated tray may be a custom replica of a subject's teeth and may aid in accurately placing the orthodontic appliance(s) on the subject's tooth (teeth). This technique is commonly referred to as indirect bonding.

In some embodiments, the low-viscosity adhesive composition, upon the application of pressure to the orthodontic appliance, may extend beyond the base perimeter to form a concave meniscus with respect to a point defined by an appliance-tooth junction. In some embodiments, the method may further include removing the low-viscosity adhesive composition from the tooth surface that has extended beyond the base perimeter. The removal of the low-viscosity adhesive composition may include brushing, wiping, picking, scraping, rinsing, or a combination thereof. In some embodiments, the minimal flash does not need to be removed.

In many embodiments, the high-viscosity adhesive composition, upon application of pressure to the orthodontic appliance, may at least partially fill a gap between the base and the tooth surface. In some embodiments, the high-viscosity adhesive composition may cover about 50 percent to about 100 percent of the total area of the base upon applying pressure to the orthodontic appliance. For example, the high-viscosity adhesive composition may cover a percentage of the total area of the base of about 50, 55, 60, 65, 70, 75, 80, 85, 90, 95, 100, or a value within a range between any of the preceding values, e.g., between about 75 and about 95, between about 80 and about 90, or the like. The contour of the tooth surface, (e.g., a molar, a cuspid, a bicuspid, or the like) may not match the contour of the base surface. The high-viscosity adhesive composition may serve to at least partially fill any gaps between the tooth surface and the base surface according to the area of the base covered upon application of pressure to the tooth surface. The base coverage may be modified according to a selection of thickness and volume of high-viscosity adhesive composition used.

In many embodiments, the high-viscosity adhesive composition, upon the application of pressure to the orthodontic appliance and upon the hardening of the hardenable adhesive layer, may not extend beyond the base perimeter by more than 0.2 mm. The high-viscosity adhesive composition may extend beyond the base perimeter in mm of less than 0.2 mm, less than 0.18, less than 0.16, less than 0.14, less than 0.12, less than 0.10, less than 0.08, less than 0.06, less than 0.04, less than 0.02, 0, or a value within a range between any of the preceding values, e.g., between about 0 and about 0.10, between about 0.04 and about 0.14, or the like.

In some embodiments, the hardening may provide a bond strength of at least 5.0 MPa as measured by the Bond Strength Test described herein. In some embodiments, the hardening may provide a bond strength in MPa of at least 5.0, at least 6.0, at least 7.0, at least 8.0, at least 9.0, at least 10.0, at least 11.0, at least 12.0, at least 13.0, at least 14.0 at least 15.0, at least 16.0, at least 17.0, at least 18.0, at least 19.0, at least 20.0, at least 21, or a values within a range between any of the preceding values, for example, between about 8.0 and about 15.0, between about 10.0 and about 20.0, or the like.

In some embodiments, the method may further include pretreating the tooth surface with a fluoride composition described below prior to contacting the orthodontic applicance to the tooth surface.

### Fluoride Pre Treatments

In some embodiments, the fluoride composition may include:
1) a fluoride-releasing composition effective to release fluoride to the tooth surface, a crosslinked polyacid polymer, a multivalent cation salt, a pharmaceutically acceptable buffer, and water;
2) a silver-fluoride composition including a source of silver cations, a source of fluoride anions, a source of iodide or thiocyanate anions, and water; and a curable resin composition including at least one (meth)acrylate monomer; or
3) a zinc carboxylate, an amine-containing ligand, a source of fluoride anion effective to provide fluoride in an amount of at least 4 wt % with respect to the weight of the fluoride composition, and water, wherein the fluoride composition has a pH of at least 8, and wherein the fluoride composition is a homogenous solution at a temperature of about 20-25° C.

In some embodiments, the method may further include pretreating the tooth surface with a fluoride composition described in 1) above. Further detail regarding the fluoride composition and method for pretreating the tooth surface can be found in WO 2019/048962, which is incorporated herein by reference in its entirety.

In some embodiments, the method may further include pretreating the tooth surface with a fluoride composition described in 2) above. Further detail regarding the fluoride composition and method for pretreating the tooth surface can be found in U.S. Prov. Pat. App. Nos. 62/956,008 and 62/968,115, each of which are incorporated by reference herein in their entireties.

In some embodiments, the method may further include pretreating the tooth surface with a fluoride composition described in 3) above. Further detail regarding the fluoride composition and method for pretreating the tooth surface can be found in U.S. Prov. Pat. App. No. 62/955,975, which is incorporated by reference in its entirety.

### Methods for Preparing Orthodontic Appliances

In various embodiments, a method for preparing an orthodontic appliance described herein is described. The method may include providing a base, and forming a hardenable adhesive layer by applying a high-viscosity adhesive composition to the base at a first region, and applying a low-viscosity adhesive composition to the base at a second region, wherein the second region at least partly surrounds the first region.

In some embodiments, the forming of the hardenable adhesive layer may include first applying the high-viscosity adhesive composition to the base at the first region, followed by applying the low-viscosity adhesive composition to the base at the second region.

In some embodiments, the applying of one or more of the high-viscosity adhesive composition and the low-viscosity adhesive composition may include extruding.

In some embodiments, the applying of one or more of the high-viscosity adhesive composition and the low-viscosity adhesive composition may include additive manufacturing practices.

In some embodiments, the applying of one or more of the high-viscosity adhesive composition and the low-viscosity adhesive composition may include manual application at the dental office.

In some embodiments, the method may further include partially hardening one or more of the high-viscosity adhesive composition and the low-viscosity adhesive composition.

In some embodiments, the method may further include shaping one or more the of the high-viscosity adhesive composition and low-viscosity adhesive composition, or otherwise removing excess adhesive from the base.

In some embodiments, the method may further include packaging the orthodontic appliance in a package described herein.

### Kits

### Kits for Use

In various embodiments, a kit is described. The kit may include an orthodontic appliance described herein and a set of instructions directing a user to perform the method steps described herein for seating the orthodontic appliance to a tooth surface.

In some embodiments, the kit may further include a package for housing the orthodontic appliance. In some embodiments, the orthodontic appliance may be suspended within the package. The package may be in the form of a blister pack. The package may further include a mount for mounting the orthodontic appliance. The mount may secure the orthodontic appliance such that the hardenable adhesive layer is protected for deformation upon handling of the package. In some embodiments, the orthodontic appliance may be packaged in a package described in WO 2019/175726, which is incorporated herein by reference in its entirety.

In some embodiments, the kit may include more than one orthodontic appliance.

### Preparation Kits

In one embodiment, a kit is described. The kit may include a base, a receptacle containing a high-viscosity adhesive composition described herein, a receptacle containing low-viscosity adhesive composition described herein, and a set of instructions directing a user to perform the method steps described herein for preparing an orthodontic appliance of the present disclosure.

In some embodiments, the kit may include more than one base.

In some embodiments, one or more of the receptacles may be graduated. A graduated compartment may allow a user to alter the amount of adhesive composition desired based on factors such as base size, tooth structure, or the like. A graduated compartment may further allow a user to apply adhesive composition to multiple bases from the same receptacle.

In some embodiments, the kit may further include one or more extruding device. In some embodiments, the extruding device(s) may be configured to mate with an adhesive composition receptacle.

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

### Materials:

HVA1—High-Viscosity Adhesive Composition #1: comprising 5-15% bisphenol A bis(2-hydroxyethyl ether) dimethacryalate, 5-15% bisphenol A diglycidyl ether dimethacryalate, 70-80% silane treated quartz, <2% silane treated silica and a camphorquinone initiator system.
HVA2—High-Viscosity Adhesive Composition #2: comprising 5-15% 2-hydroxy-1,2,3-propanetricarboxylic acid reaction products with 2-isocyanatoethyl methacrylate, 5-15% polyethylene glycol dimethacrylate, 1-10% bisphenol A diglycidyl ether dimethacryalate, 35-45% silane treated glass, 35-45% silane treated quartz, 1-5% silane treated silica and a camphorquinone initiator system.
LVA1—Low-Viscosity Adhesive Composition #1: comprising 44% bisphenol A bis(2-hydroxyethyl ether) dimethacryalate, 44% bisphenol A diglycidyl ether dimethacryalate, 11% silane treated ceramic, 0.25% camphorquinone, 1% ethyl 4-dimethylaminobenzoate, 0.15% diphenyliodonium hexafluorophosphate and 0.1% butylated hydroxytoluene (viscosity of approximately 20-100 Pa·s).
LVA2—Low-Viscosity Adhesive Composition #2: comprising 40-50% triethylene glycol dimethacrylate, 40-50% bisphenol A diglycidyl ether dimethacryalate, 6% silane treated fumed silica, <5% tetrabutylammonium tetrafluoroborate, <0.5% titanium dioxide, <0.05% Rose Bengal dye, and an initiator system based on camphorquinone, a tertiary amine, and an iodonium salt (viscosity of approximately between 0.5-5 Pa·s).
LVA3—Low-Viscosity Adhesive Composition #3: comprising 15-25% 2-hydroxyethyl methacrylate, 15-25% bisphenol A diglycidyl ether dimethacryalate, 5-15% decamethylene dimethacrylate, 1-10% methacryloxydecyl phosphate, <2% dimethylaminoethyl methacrylate, 1-5% copolymer of acrylic acid and itaconic acid, 5-15% silane treated silica, 10-15% ethanol, 10-15% water, and a camphorquinone initiator system (viscosity of approximately between 0.1-1 Pa-s).

Nonwoven mat used in a comparative example was a compressible material composed of polypropylene with individual fibers of diameter approximately 6 microns.

### Viscosity Measurement

The viscosities of high-viscosity adhesives were tested using a controlled strain rheometer (Model ARG2, TA Instruments, Eden Prairie, MN). Adhesive samples were placed in between two parallel plates (8 mm diameter) at a gap of 0.15 mm. Viscosity measurements were performed at shear rates starting from 0.01 s⁻¹ to 10 s⁻¹ at 25° C. in 23 logarithmically spaced shear rate steps. Prior to measurements, samples were pre-sheared at 10 s⁻¹ for 3 minutes and allowed to equilibrate for 5 minutes at 25° C.

| High- | Viscosity at | Viscosity at | Viscosity at |
|---|---|---|---|
| Viscosity | (Pa-s) 0.1 s⁻¹ | (Pa-s) 1 s⁻¹ | (Pa-s) 10 s⁻¹ |
| Adhesive | shear rate | shear rate | shear rate |
| HVA1 | 6441 | 4164 | 1577 |
| HVA2 | 5809 | 1879 | 450 |

The viscosities of low-viscosity adhesives were tested using a controlled strain rheometer (Model ARG2, TA Instruments, Eden Prairie, MN). Adhesive samples were placed in between two parallel plates (40 mm diameter) at a gap of 0.50 mm. Viscosity measurements were performed at shear rates starting from 0.01 s⁻¹ to 200 s⁻¹ at 25° C. in 23 logarithmically spaced shear rate steps. Prior to measurements, samples were pre-sheared at 10 s⁻¹ for 3 minutes and allowed to equilibrate for 5 minutes at 25° C.

| Low- | Viscosity at | Viscosity at | Viscosity at |
|---|---|---|---|
| Viscosity | (Pa-s) 0.1 s⁻¹ | (Pa-s) 1 s⁻¹ | (Pa-s) 10 s⁻¹ |
| Adhesive | shear rate | shear rate | shear rate |
| LVA1 | 33.6 | 34.3 | 29.7 |
| LVA2 | 1.11 | 0.99 | 1.09 |
| LVA3 | 0.96 | 0.78 | 0.71 |

### Comparative Example CE1: Bracket with High-Viscosity Adhesive Composition #1 (HVA1)

Comparative Example 1 was commercially available product 3M™ SmartClip™ SL3 Self-Ligating Brackets with APC™ II Adhesive precoat (Catalog number 3004-301), commercially available from 3M Company of St. Paul, MN, USA). The APC™ II Adhesive is a high-viscosity paste adhesive, referred to as HVA1.

### Comparative Example CE2: Bracket with Only High-Viscosity Adhesive Composition #2 (HVA2)

Comparative Example 2 was commercially available product SmartClip™ SL3 with APC™ PLUS Adhesive precoat (Catalog number 5004-301), commercially available from 3M Company of St. Paul, MN, USA). The APC™ PLUS Adhesive is another high-viscosity paste adhesive, referred to as HVA2.

### Comparative Example CE3: Bracket with Only Low-Viscosity Adhesive Composition (LVA1)

Comparative Example 3 was prepared by coating the base of commercially available orthodontic bracket SmartClip™ SL3 (3M Catalog number 004-301) with a low-viscosity adhesive (LVA1) described under Materials.

### Example Ex.1: Bracket with High-Viscosity Adhesive Composition #1 (HVA1) Surrounded by Low-Viscosity Adhesive (LVA1)

Example 1 was prepared by modifying the commercially available 3M™ SmartClip™ SL3 Self-Ligating Brackets with APC™ II Adhesive precoat (Catalog number 3004-301), of Comparative Example 1. The high-viscosity paste adhesive (HVA1) around the periphery of the bracket base was removed using a razor blade, leaving an inner central portion of the bracket base with APC™ II Adhesive. Low-viscosity adhesive (LVA1) was then applied to the bracket base around the remaining inner portion of HVA1. The low-viscosity adhesive (LVA1) used was described under Materials. The weight of HVA1 was 4.2 mg (68 wt %, 54 vol %) and that of LVA1 was 2.0 mg (32 wt %, 46 vol %).

### Example Ex.2: Bracket with High-Viscosity Adhesive Composition #2 (HVA2) Surrounded by Low-Viscosity Adhesive (LVA1)

Example 2 was prepared by modifying the commercially available product SmartClip™ SL3 with APC™ PLUS Adhesive precoat (Catalog number 5004-301), of Comparative Example 2. The high-viscosity paste adhesive (HVA2) around the periphery of the bracket base was removed using a razor blade, leaving an inner central portion of the bracket base with APC™ PLUS Adhesive. Low-viscosity adhesive (LVA1) was then applied to the bracket base around the remaining inner portion of HVA2. The low-viscosity adhesive (LVA1) used was described under Materials. The weight of HVA2 was 4.2 mg (68 wt %, 54 vol %) and that of LVA1 was 2.0 mg (32 wt %, 46 vol %).

### Bonding Procedure

Bovine teeth were cleaned and partially embedded in circular polymethylmethacrylate discs with the labial tooth surface exposed. The teeth were prophied, etched and primed with TRANSBOND Plus Self Etching Primer (SEP) (Commercially available from 3M Company, Catalog number 712-090) for 3-5 sec and air blown dry for 3 seconds. The precoated brackets were bonded to bovine teeth. Upon seating brackets to the teeth, adhesive flash (excess) cleanup was not needed for Examples 1-2 (with LVA at the outer region) and Comparative Example 3 (with LVA only). However, per standard clinical bracket bonding practice, the excessive adhesive flash around the base of the brackets was removed for Comparative Examples 1-2, which had only HVA paste adhesives. Ortholux™ Luminous Curing Light (available from 3M Company of St. Paul, MN, USA; catalog number 704-460) was used to light cure all the adhesives in Examples 1-2 and Comparative Examples 1-3; the curing light was shined on the examples for 3 seconds mesial, and 3 seconds distal unless otherwise noted.

### Shear Peel Bond Strength Test Procedure

Shear peel bond strength was determined either at 15 minutes after bonding or 16-24 hours after bonding using the following standardized method. First, each bonded test specimen was mounted with the gingival tie wings oriented upward (unless otherwise noted) in a test fixture attached to a QTEST/5 brand mechanical testing machine (MTS Systems Corporation, Eden Prairie, Minn.). A 0.020 inch (0.051 centimeter) diameter standard round wire was looped under the occlusal tie wings and attached to the crosshead of the testing machine. After initial crosshead position was adjusted to make the wire snug, it was translated upward at 0.2 inches per minute (5 millimeters per minute) until the bracket was debonded. Maximum force was recorded and divided by the measured surface area of the bracket base to obtain a bond strength measurement. Each reported bond strength value represents an average of ten replicated measurements unless otherwise noted.

TABLE 1
Bond strengths of Comparative Examples 1-3 and Examples 1-2

| | | | Average | Average |
|---|---|---|---|---|
| | Bond | Adhesive on | Bond | Bond |
| | strength | SmartClip ™ | strength | strength |
| Example | at | SL3 | (lb/bracket) | (MPa) |
| Comparative CE1 | 15 min | HVA1 only | 33.0 | 13.9 |
| Comparative CE2 | 15 min | HVA2 only | 54.6 | 23.0 |
| Comparative CE3 | 15 min | LVA1 only | 19.8 | 8.3 |
| Example Ex. 1 | 15 min | HVA1 | 36.4 | 15.4 |
| | | surrounded | | |
| | | by LVA1 | | |
| Example Ex. 2 | 15 min | HVA2 | 51.4 | 21.7 |
| | | surrounded | | |
| | | by LVA1 | | |

Examples Ex.1 and Ex.2 exhibited acceptable bond strength, compared to commercial products Comparative Examples CE1 and CE2. Furthermore, Examples Ex.1 and Ex.2 also had the benefit of no need for adhesive flash cleanup since the periphery LVA1 adhesive resulted in a low profile/minimal flash.

### Example Ex.3: Buccal Tubes with High-Viscosity Adhesive #1 (HVA1) Surrounded by Low-Viscosity Adhesive (LVA1)

Example 3 was prepared by modifying the commercially available 3M product: Victory Series™ buccal tubes with APC™ II Adhesive precoat (3M catalog number 3066-4082). The high-viscosity paste adhesive (HVA1) already present on the commercially available product was partially removed in the region around the periphery of the base using a razor blade, leaving an inner central portion of the base covered with HVA1 (APC™ II adhesive). Low-viscosity adhesive (LVA1) was then applied to the base around the remaining inner portion of HVA1. The low-viscosity adhesive (LVA1) used was described under Materials. The weight of HVA1 was 9.6 mg (82 wt %, 72 vol %) and that of LVA1 was 2.1 mg (18 wt %, 28 vol %).

### Comparative Example CE4: Buccal Tube with High-Viscosity Adhesive #1 (HVA1)

Comparative Example 4 was commercially available 3M product: Victory Series™ buccal tubes with APC™ II Adhesive precoat (3M catalog number 3066-4082). The APC™ II adhesive present on this product is a high-viscosity paste adhesive, referred to as HVA1.

### Comparative Example CE5: Buccal Tube with Low-Viscosity Adhesive (LVA1)

Comparative Example 5 was prepared by coating the base of the commercially available 3M Victory Series™ buccal tubes (3M catalog number 066-4082) with only a low-viscosity adhesive (LVA1) described under Materials section.

### Comparative Example CE6: Buccal Tube with Low-Viscosity Adhesive (LVA1) with Nonwoven Mat

Comparative Example 6 was prepared in the same manner as Comparative Example CE5, except additionally, a nonwoven mat described under Materials was also cut to the shape of the base and installed on the base of the buccal tube. The nonwoven mat was completely saturated with the low-viscosity adhesive (LVA1) described under Materials. Upon seating the appliance, the low-viscosity adhesive (LVA1) seeped out to fill the gap between the appliance base and tooth surface and form a meniscus around the edges of the base, thereby eliminating the need for excess adhesive flash removal.

In the Bonding Procedure, adhesive flash (excess) cleanup was not needed for Example 3 (with LVA at eh outer region), Comparative Example 5 (with LVA only) and Comparative Example 6 (LVA and nonwoven mat). However, per standard clinical bracket bonding practice, the excessive adhesive flash around the base of the buccal tubes was removed for Comparative Example 4, which had only HVA paste adhesives. The curing time used for buccal tube examples in Table 2 was 6 seconds mesial and 6 seconds occlusal.

In the Shear-Peel Bond Strength Test Procedure, these buccal tube examples in Table 2 were debonded with the hook oriented downward.

TABLE 2
Bond strengths of Comparative Examples 4-6 and Example 3

| | | | Average | Average |
|---|---|---|---|---|
| | Bond | Adhesive on | Bond | Bond |
| | strength | Victory Series ™ | strength | strength |
| Example | at | buccal tubes | (lb/bracket) | (MPa) |
| Comparative CE4 | 15 min | HVA1 only | 31.0 | 13.0 |
| Comparative CE5 | 15 min | LVA1 only | 21.4 | 9.0 |
| Comparative CE6 | 15 min | LVA1 saturated | 22.5 | 9.5 |
| | | nonwoven | | |
| Example Ex. 3 | 15 min | HVA1 surrounded | 30.0 | 12.6 |
| | | by LVA1 | | |

Example Ex.3 exhibited acceptable bond strength, compared to commercial product Comparative Example CE4. Furthermore, Example Ex.3 did not require adhesive flash cleanup since the periphery LVA1 adhesive resulted in a low profile/minimal flash.

### Example Ex.4: Low Profile Bracket with High-Viscosity Adhesive #1 (HVA1) Surrounded by Low-Viscosity Adhesive (LVA1)

Example 4 was prepared by modifying the commercially available 3M product: Victory Series™ Low Profile bracket with APC™ II Adhesive precoat (3M catalog number 3024-890). The high-viscosity paste adhesive (HVA1) already present on the commercially available product was partially removed in the region around the periphery of the base using a razor blade, leaving an inner central portion of the base covered with HVA1. Low-viscosity adhesive (LVA1) was then applied to the base around the remaining inner portion of HVA1. The low-viscosity adhesive (LVA1) used was described under Materials. The weight of HVA1 was 3.5 mg (69 wt %, 55 vol %) and that of LVA1 was 1.6 mg (31 wt %, 45 vol %).

### Vertical Flow Test

Five replicates of Example 4 were prepared and tested for vertical flow of adhesive. The brackets were positioned so that the bases were vertical (90 degrees to horizontal). After 6 hours at room temperature the profiles of the Example 4 brackets were observed. No flow of adhesive (HVA1 or LVA1) was observed; there was no change in the thickness of the adhesive at the top of the bracket vs the bottom of the bracket. The Example 4 brackets were then subjected to 5 minutes at 40° C. (also in the vertical position). Again, there was no (undesirable) adhesive flow observed. Additionally, the viscosity difference between HVA1 and LVA1 was large enough that they remained separate and did not mix into one.

### Comparative Example 7: Low Profile Bracket with High-Viscosity Adhesive #1 (HVA1)

Comparative Example 7 was commercially available 3M product: Victory Series™ Low Profile Brackets with APC™ II Adhesive precoat (3M catalog number 3024-890). The APC™ II Adhesive present on this product is a high-viscosity paste adhesive, referred to as HVA1.

### Comparative Example 8: Low Profile Bracket with Low-Viscosity Adhesive #1 (LVA1)

Comparative Example 8 was prepared by coating the base of commercially available orthodontic bracket Victory Series™ Low Profile (3M Catalog number 024-890) with a low-viscosity adhesive (LVA1) described under Materials.

In the Bonding Procedure, adhesive flash (excess) cleanup was not needed for Example 4 (with LVA at the outer/second region) and Comparative Example 8 (with LVA only). However, per standard clinical bracket bonding practice, the excessive adhesive flash around the base of the brackets was removed for Comparative Example 7, which had only HVA paste adhesives. In the Shear-Peel Bond Strength Test Procedure, examples of this type of brackets in Table 3 were debonded with gingival tie wings oriented downward.

TABLE 3
Bond strengths of Comparative Examples 7-8 and Example 4

| | | | Average | Average |
|---|---|---|---|---|
| | Bond | Adhesive on | Bond | Bond |
| | strength | Victory Series ™ | strength | strength |
| Example | at | Low Profile | (lb/bracket) | (MPa) |
| Comparative CE7 | 15 min | HVA1 only | 24.7 | 13.9 |
| Comparative CE8 | 15 min | LVA1 only | 20.1 | 12.1 |
| Example Ex. 4 | 15 min | HVA1 surrounded | 28.5 | 9.8 |
| | | by LVA1 | | |

Example Ex.4 exhibited acceptable bond strength, compared to commercial product Comparative Example CE7. Furthermore, Example Ex.4 did not require adhesive flash cleanup since the periphery LVA1 adhesive resulted in a low profile/minimal flash.

### Example Ex.5: Low Profile Bracket with High-Viscosity Adhesive Composition #1 (HVA1) Surrounded by Low-Viscosity Adhesive (LVA2)

Example 5 was prepared by modifying the commercially available 3M product: Victory Series™ Low Profile bracket with APC™ II Adhesive precoat (3M catalog number 3024-875). The high-viscosity paste adhesive (HVA1) already present on the commercially available product was partially removed in the region around the periphery of the base using a razor blade, leaving an inner central portion of the base covered with HVA1. Low-viscosity adhesive (LVA2) was then applied to the base around the remaining inner portion of HVA1. The weight of HVA1 was 4.0 mg (68 wt %, 55 vol %) and that of LVA2 was 1.9 mg (32 wt %, 45 vol %).

### Comparative Example 9: Low Profile Bracket with High-Viscosity Adhesive Composition #1 (HVA1)

Comparative Example 9 was commercially available 3M product: APC II Victory Series™ Low Profile Brackets (3M catalog number 3024-875). The APC™ II Adhesive present on this product is a high-viscosity paste adhesive, referred to as HVA1.

### Comparative Example 10: Low Profile Bracket with Low-Viscosity Adhesive Composition #2 (LVA2)

Comparative Example 10 was prepared by coating the base of commercially available orthodontic bracket Victory Series™ Low Profile (3M Catalog number 024-875 or 024-775) with 3M CLINPRO Sealant, commercially available from 3M Company of St. Paul, MN, USA). The 3M CLINPRO Sealant is a low-viscosity adhesive, referred to as LVA2.

In the Bonding Procedure, adhesive flash (excess) cleanup was not needed for Example 5 (with LVA at the outer/second region) and Comparative Example 10 (with LVA only). However, per standard clinical bracket bonding practice, the excessive adhesive flash around the base of the brackets was removed for Comparative Example 9, which had only HVA paste adhesives. In Table 4, the reported Shear Peel Bond Strength Test value represents an average of ten replicated measurements in Comparative Example 9 or an average of eight replicated measurements in Example 5 and Comparative Example 10.

TABLE 4
Bond strengths of Comparative Examples 9-10 and Example 5

| | | | Average | Average |
|---|---|---|---|---|
| | Bond | Adhesive on | Bond | Bond |
| | strength | Victory Series ™ | strength | strength |
| Example | at | Low Profile | (lb/bracket) | (MPa) |
| Comparative CE9 | 15 min | HVA1 only | 34.4 | 14.5 |
| Comparative CE10 | 15 min | LVA2 only | 20.0 | 8.4 |
| Example Ex. 5 | 15 min | HVA1 | 39.3 | 16.6 |
| | | surrounded | | |
| | | by LVA2 | | |

Example Ex.5 exhibited acceptable bond strength, compared to commercial product Comparative Example CE9. Furthermore, Example Ex.5 did not require adhesive flash cleanup since the periphery LVA2 adhesive resulted in a low profile/minimal flash.

### Example Ex.6: Low Profile Bracket with High-Viscosity Adhesive Composition #1 (HVA1) Surrounded by Low-Viscosity Adhesive (LVA3)

Example 6 was prepared by modifying the commercially available 3M product: Victory Series™ Low Profile bracket with APC™ II Adhesive precoat (3M catalog number 3024-890). The high-viscosity paste adhesive (HVA1) already present on the commercially available product was partially removed in the region around the periphery of the base using a razor blade, leaving an inner central portion of the base covered with HVA1. Low-viscosity adhesive (LVA3) was then applied to the base around the remaining inner portion of HVA1. The low-viscosity adhesive (LVA3) used was a self-etching adhesive: Scotchbond Universal Adhesive (3M catalog number 41528) with a pH of 2.7. The weight of HVA1 was 3.6 mg (69 wt %, 55 vol %) and that of LVA3 was 1.6 mg (31 wt %, 45 vol %). An air syringe was used to dry the solvents for 5 seconds after the application of LVA3. In the Bonding Procedure, etching and priming the tooth with TRANSBOND Plus Self Etching Primer (SEP) was omitted.

### Comparative Example 11: Low Profile Bracket with High-Viscosity Adhesive Composition #1 (HVA1)

Comparative Example 11 was commercially available 3M product: Victory Series™ Low Profile Brackets with APC II Adhesive precoat (3M catalog number 3024-890). The APC™ II Adhesive present on this product is a high-viscosity paste adhesive, referred to as HVA1.

In the Bonding Procedure, adhesive flash (excess) cleanup was not needed for Example 6 (with LVA at the outer/second region). However, per standard clinical bracket bonding practice, the excessive adhesive flash around the base of the brackets was removed for Comparative Example 11, which had only HVA paste adhesives. In the Shear-Peel Bond Strength Test Procedure, examples of this type of brackets in Table 5 (same as those used in Table 3) were debonded with gingival tie wings oriented downward.

TABLE 5
Bond strengths of Comparative Example 11 and Example 6

| | | | Adhesive | Average | Average |
|---|---|---|---|---|---|
| | Bond | Etchant/ | on Victory | Bond | Bond |
| | strength | primer | Series ™ | strength | strength |
| Example | at | on tooth | Low Profile | (lb/bracket) | (MPa) |
| Comparative | 24 hr | SEP | HVA1 only | 39.3 | 19.2 |
| CE11 | | | | | |
| Example | 24 hr | none | HVA 1 | 34.1 | 16.7 |
| Ex. 6 | | | surrounded | | |
| | | | by LVA3 | | |

Example Ex.6 exhibited acceptable bond strength, compared to commercial product Comparative Example CE11. Example Ex.6 eliminated the need for etching/priming the tooth as a result of the acidic function of the low-viscosity adhesive LVA3. Furthermore, Example Ex.6 did not require adhesive flash cleanup since the periphery low-viscosity adhesive LVA3 resulted in a low profile/minimal flash.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. An orthodontic appliance comprising:
a base; and
an hardenable adhesive layer disposed on the base,
the adhesive layer comprising a first region and a second region,
the first region comprising a high-viscosity adhesive composition characterized by having a viscosity of about 10 Pa·s to about 1,500,000 Pa·s at a shear rate of 1 s⁻¹, and
the second region comprising a low-viscosity adhesive composition characterized by having a viscosity of about 0.1 Pa·s to about 100 Pa·s at a shear rate of 1 s⁻¹,
wherein the first region is at least partly surrounded by the second region,
wherein the first region and the second region are configured to contact a tooth surface, and
wherein the viscosity of the high-viscosity adhesive composition is greater than the viscosity of the low-viscosity adhesive composition.

2. The orthodontic appliance of claim 1, wherein the high-viscosity adhesive composition does not extend beyond the base by more than 0.2 mm when the hardenable adhesive layer is compressed against the tooth surface.

3. The orthodontic appliance of claim 1, wherein the high-viscosity adhesive composition comprises a filler present in an amount of about 50 to about 86 wt % with respect to the weight of the high-viscosity adhesive.

4. (canceled)

5. (canceled)

6. (canceled)

7. The orthodontic appliance of claim 1, wherein one or more of:
the high-viscosity adhesive composition is characterized by a static yield stress at 28° C. of at least 7000 dynes/cm²; and
the low-viscosity adhesive composition is characterized by a flow where the difference of the thicknesses of the adhesive composition at the top and at the bottom is less than about 15%, as determined by the Vertical Flow Test.

8. The orthodontic appliance of claim 1, wherein the high-viscosity adhesive composition has a thickness of about 0.38 mm to about 1.2 mm.

9. The orthodontic appliance of claim 1, wherein the hardenable adhesive layer is in a shape representing a gaussian curve, a triangle, or trapezoid when viewed from a cross-sectional side view of the base.

10. The orthodontic appliance of claim 1, wherein the high-viscosity adhesive composition is in the shape representing a circle, a polygon, or a quadrilateral when viewed from a direction perpendicular to the base.

11. The orthodontic appliance of claim 1, wherein the high-viscosity adhesive composition has a volume that is about 40 percent to about 85 percent of the volume of the hardenable adhesive layer.

12. The orthodontic appliance of claim 1, wherein the high-viscosity adhesive composition extends over about 20 percent to about 50 percent of the total area of the base.

13. (canceled)

14. The orthodontic appliance of claim 1, wherein the low-viscosity adhesive composition has a filler loading of about 0 to about 50 wt %.

15. (canceled)

16. (canceled)

17. The orthodontic appliance of claim 1, wherein the low-viscosity adhesive composition has a thickness of about 0.06 mm to about 0.16 mm.

18. The orthodontic appliance of claim 1, wherein the low-viscosity adhesive composition is in the shape of an annulus or rectangular annulus when viewed from a direction perpendicular to the base.

19. The orthodontic appliance of claim 1, wherein the low-viscosity adhesive composition extends over about 50 percent to about 80 percent of the total area of the base.

20. (canceled)

21. The orthodontic appliance of claim 1, wherein one or more of:
the high-viscosity adhesive composition is characterized by a viscosity of about 100 Pa·s to about 13,000 Pa·s at a shear rate of 1 s⁻¹; and
the low-viscosity adhesive composition is characterized by a viscosity of about 0.1 Pa·s to about 10 Pa·s at a shear rate of 1 s⁻¹.

22. (canceled)

23. The orthodontic appliance of claim 1, wherein the high-viscosity adhesive composition and the low-viscosity adhesive composition differ in viscosity by at least 10-fold.

24. The orthodontic appliance of claim 1, wherein the high-viscosity adhesive composition has a maximum thickness that is greater than an average thickness of the low-viscosity adhesive composition.

25. (canceled)

26. The orthodontic appliance of claim 1, being void of a compressible mat.

27. A method for seating an orthodontic appliance to a tooth surface, the method comprising:
providing an orthodontic appliance of claim 1;
contacting the orthodontic appliance to the tooth surface;
applying pressure to the orthodontic appliance such that the hardenable adhesive layer compresses against the tooth surface; and
hardening the hardenable adhesive layer to form a hardened adhesive.

28. (canceled)

29. (canceled)

30. (canceled)

31. (canceled)

32. (canceled)

33. (canceled)

34. (canceled)

35. (canceled)

36. (canceled)

37. (canceled)

38. A kit comprising:
an orthodontic appliance of claim 1; and
a set of instructions directing a user to seat the orthodontic appliance to a tooth surface; or
a base;
a receptacle comprising a high-viscosity adhesive composition therein;
a receptacle comprising a low-viscosity adhesive composition therein; and
a set of instructions directing a user to prepare the orthodontic appliance.

39. (canceled)

40. (canceled)

41. A method of preparing an orthodontic appliance of claim 1, the method comprising:
providing a base; and
forming a hardenable adhesive layer on the base, the forming comprising:
applying a high-viscosity adhesive composition to the base at a first region, and
applying a low-viscosity adhesive composition to the base at a second region,
wherein the first region is at least partly surrounded by the second region.

42. (canceled)

43. (canceled)

44. (canceled)
